(19) United States
(12) Patent Application Publication
(10) Pub. No.: US 2025/0130221 A1
(43) Pub. Date: Apr. 24, 2025

(54) METHOD OF IDENTIFYING AGENTS FOR THE TREATMENT OF CYSTIC FIBROSIS CAUSED BY THE MUTATION F508DEL

(71) Applicant: FACULDADE DE CIÊNCIAS DA UNIVERSIDADE DE LISBOA, LISBOA (PT)

(72) Inventors: Margarida DE AMARAL, LISBOA (PT); Hugo Miguel RAPOSO CORREIA BOTELHO, BOBADELA LRS (PT); Miquéias LOPES-PACHECO, LISBOA (PT)

(21) Appl. No.: 18/835,591

(22) PCT Filed: Feb. 27, 2023
(86) PCT No.: PCT/IB2023/051813

§ 371 Date: Aug. 2, 2024

(30) Foreign Application Priority Data
Feb. 25, 2022 (PT) 117811

(51) Int. Cl. G01N 33/50; A61K 31/405; A61P 43/00; G01N 33/68

(52) U.S. Cl. CPC G01N 33/5023; A61K 31/405; A61P 43/00; G01N 33/6893; G01N 2333/4703; G01N 2333/91215; G01N 2333/9124; G01N 2500/10

(57) ABSTRACT

A method of identifying agents for the treatment of Cystic Fibrosis (CF) caused by the mutation F508del in the gene encoding the cystic fibrosis transmembrane conductance regulator protein (F508del-CFTR), wherein a candidate agent contacts with at least one gene sequence selected from SEQ ID NOs: 228 to 478 or with its protein product as therapeutic targets. The ability of the candidate agent to modulate the activity of the F508del-CFTR protein and its plasma membrane traffic is determined. Gene sequences SEQ ID NOs: 228 to 478 were identified by a high-throughput siRNA cell-based microscopy screen aimed at identifying genes which enhance F508del-CFTR plasma membrane levels upon knock-down, as potential candidates for drug therapy in the treatment of CF. Further disclosing formulations comprising agents capable of targeting at least one gene sequence selected from SEQ ID NOs: 228 to 478 or their protein product.

### TECHNICAL FIELD

This application relates to a method of identifying agents for the treatment of Cystic Fibrosis caused by the mutation F508del in the gene encoding the cystic fibrosis transmembrane conductance regulator protein (F508del-CFTR), and compositions comprising said agent.

### BACKGROUND ART

About 80% of individuals with Cystic Fibrosis (CF), a life-shortening disease affecting >90,000 individuals worldwide predominantly with respiratory symptoms, have F508del. This mutation causes the CF transmembrane conductance regulator (CFTR) protein to misfold and being targeted by the endoplasmic reticulum (ER) quality control (ERQC) for premature degradation, thus preventing its plasma membrane (PM) traffic. Despite the recent approval of a ‘highly effective’ drug combination rescuing F508del-CFTR, maximal lung function improvement is ˜14% and the drug-targeted genes remain unknown.

Mutations in the gene encoding the Cystic Fibrosis Transmembrane Conductance Regulator (CFTR) protein (Riordan et al., 1989) cause Cystic Fibrosis (CF), the most common life-shortening autosomal recessive disease (median age at death ˜37 years (MacKenzie et al., 2014)) affecting >90,000 individuals worldwide. CFTR is a chloride (Cl⁻) and bicarbonate (HCO₃⁻) channel expressed at the apical membrane of several epithelial tissues, notably in the airways, which are the most affected organ in CF. In the airways, CFTR maintains epithelial ion homeostasis and fluid secretion/hydration of the airway surface liquid (ASL) and dysfunctional CFTR leads to ASL dehydration and persistence of an immovable thick mucus which obstructs the airway and prevents mucociliary clearance (MCC) thus potentiating chronic inflammation and recurrent bacterial infections that, over time, lead to progressive deterioration of lung function, the most frequent cause of morbidity and mortality in CF.

Among the >2,100 CFTR mutations so far reported (2021), the deletion of residue phenylalanine 508—F508del—is the most common, occurring in ˜80% of CF cases worldwide, being thus a preferential target for research and drug development in CF. F508del-CFTR presents several defects, namely: i) protein misfolding which is resilient to physiological rescue by endogenous molecular chaperones; ii) deficient PM traffic due to retention of misfolded F508del-CFTR at several checkpoints of the ER quality control (ERQC) machinery (Amaral, 2004) which target it for premature degradation via the ubiquitin (Ub)-proteasomal pathway (UPP); iii) impaired function, as the residual amount of F508del-CFTR that reaches the PM (only in some patients) has very low activity, due to a defect in the channel gating (Dalemans et al., 1991); iv) PM instability of rescued F508del-CFTR as it is quickly endocytosed and degraded (Moniz et al., 2013; Okiyoneda et al., 2013).

Nevertheless, early studies have shown that F508del-CFTR can be released from the ER to the PM, namely by low temperature incubation of cells heterologously expressing the mutant (Denning et al., 1992). This was valid proof-of-principle to demonstrate that F508del-CFTR ER retention can be rescued. However, only recently a triple drug, combining two correctors—tezacaftor and elexacaftor—to rescue the PM traffic of F508del-CFTR with a potentiator—ivacaftor—to rescue the gating defect in individuals with CF and at least one F508del mutation was shown to be efficacious (Middleton et al., 2019; Taylor-Cousar et al., 2017)). Nevertheless, the maximum clinical benefit of this ‘highly effective’ triple combo drug is an increase in lung function by only ˜14%, with some variability, likely due to the influence of modifier genes modulating the efficacy of the response to the drug.

Despite evidence that one of these correctors (tezacaftor) binds to mutant CFTR (Farinha et al., 2013a; He et al., 2013; Lopes-Pacheco et al., 2020; Okiyoneda et al., 2013; Fiedorczuk et al., 2022), the mechanism of action (MoA) for the second corrector (elexacaftor) remains unknown. Thus, identifying the multiple intervenients assessing CFTR folding at the ERQC checkpoints (Amaral and Farinha, 2013; Farinha and Amaral, 2005) will likely shed some light into the MoA of this and future traffic-rescuing drugs (Amaral, 2021) being crucial for the development of rationale and therapeutic strategies that significantly increase the efficacy of F508del-CFTR rescue. This strategy may also enable rescue of other folding CFTR mutants, and/or numerous misfolded proteins causing traffic disorders (Yarwood et al., 2020).

The complex ERQC process by which CFTR folding is assessed has been proposed to consist in main four sequential checkpoints (Amaral et al., 2016; Farinha and Amaral, 2005; Farinha and Canato, 2017; Farinha et al., 2013a; Farinha et al., 2013b; Younger et al., 2006), namely through: 1) interaction with the Hsp70 chaperone machinery where most F508del-CFTR is degraded; 2) the calnexin folding cycle; 3) exposure to an as yet unidentified machinery of arginine-framed tripeptides (AFT: Arg-X-Arg), four of which are present in CFTR; 4) export from the ER exit sites into coat protein (COP) II-coated vesicles mediated by a di-acidic exit code (Asp565-Ala-Asp567) present in CFTR (Nishimura and Balch, 1997; Wang et al., 2004). Failure to overcome one or more of these checkpoints inevitably leads to CFTR retention in the ER and subsequent degradation via UPP.

Most of the knowledge on these ERQC checkpoints has relied on studies with second-site “revertant” mutations which render F508del-CFTR less susceptible to UPP at each of these individual checkpoints (Amaral and Farinha, 2013). These revertants have also enabled dissecting the mechanism of action of corrector drugs (Farinha et al., 2013a). Nevertheless, the factors that retain F508del-CFTR at each of the above ERQC checkpoints have only been partially elucidated by hypothesis-driven approaches, remaining the global cellular machinery responsible for the ERQC of F508del-CFTR still unveiled.

### SUMMARY

The present invention relates to a method of identifying agents for the treatment of Cystic Fibrosis caused by the mutation F508del in the gene encoding the transmembrane conductance regulator protein (F508del-CFTR) comprising the steps of:
a) contacting a candidate agent with at least one gene sequence selected from Seq.ID: 228 to 478 or with at least one of its protein products of Seq.ID: 1 to 227 as therapeutic targets;
b) determining whether the candidate agent modulates the activity of the F508del-CFTR protein and its plasma membrane traffic.

In one embodiment at least one gene sequence is selected from Seq.ID: 232, 237, 238, 250, 253, 257, 259, 263, 276, 285, 296, 297, 301, 317, 318, 319, 333, 338, 369, 378, 396, 401, 405, 409, 410, 411, 417, 420, 421, 430, 442, 450, 458, 459, 471, 472 and 478.

In one embodiment the activity modulation is affected by inhibiting gene expression of at least one gene sequence selected from Seq.ID: 228 to 478 or the activity of at least one of its protein products of Seq.ID: 1 to 227.

In one embodiment the candidate agents identified inhibit the activity of at least one gene sequence selected from Seq.ID: 228 to 478 or the activity of at least one of its protein products of Seq.ID: 1 to 227 by more than 20% relative to the activity in the absence of the candidate agent.

In one embodiment the inhibition is affected by reducing gene expression of at least one gene sequence selected from Seq.ID: 228 to 478.

In one embodiment the activity is inhibited on the protein level of at least one protein sequence selected from Seq.ID: 1 to 227.

In one embodiment the ability of the candidate agent to bind to a protein product, or a domain thereof, from at least one gene sequence selected from Seq.ID: 228 to 478 or from at least one protein sequence from Seq.ID: 1 to 227 is determined.

In one embodiment the gene sequence protein products are TPK1, LRRK1, STYK1, GRK5 or DGKG.

In one embodiment (R,Z)-3-((4-(2-Bromoacetamido)-3,5-dimethyl-1H-pyrrol-2-yl)-methylene)-2-oxo-N-(1-phenylethyl)indoline-5-carboxamide is the candidate agent.

In one embodiment the method is carried out as a high-throughput assay.

In one embodiment the method is carried out as a cell-based assay.

The present invention also relates to an agent for use in the treatment of Cystic Fibrosis caused by the mutation F508del in the gene encoding the CFTR protein identified by the method.

In one embodiment the agent is (R,Z)-3-((4-(2-Bromoacetamido)-3,5-dimethyl-1H-pyrrol-2-yl)-methylene)-2-oxo-N-(1-phenylethyl)indoline-5-carboxamide.

The present invention also relates to a pharmaceutical composition comprising the agent identified by the method.

In one embodiment the pharmaceutical composition further comprises at least one pharmaceutically acceptable diluent, carrier, adjuvant or an excipient.

### GENERAL DESCRIPTION

The present invention relates to a method of identifying agents for the treatment of Cystic Fibrosis caused by the mutation F508del in the gene encoding CFTR.

To identify global modulators of F508del traffic, a high-content siRNA microscopy-based screen of >9,000 genes and monitorization F508del-CFTR PM rescue in human airway cells was performed. This primary screen identified the protein products of 227 genes as F508del-CFTR traffic regulators, of which at least 35 were validated by additional siRNAs, being thus further characterized. Subsequent mechanistic studies established GRK5 as a robust regulator whose inhibition rescues F508del-CFTR PM traffic, thus emerging as a novel potential drug target for CF.

There still exist unknown cellular factors that retain F508del-CFTR in the ER. A High-Throughput (HT) microscopy platform combined with siRNA knock-down (KD) to screen for global candidate regulators of F508del-CFTR PM traffic was applied with the ultimate goal of discovering novel potential drug targets for CF. To this end, a previously reported cell-based microscopy traffic assay combined with siRNA KDs was used to screen a library of 27,312 siRNAs by HT fluorescence microscopy (Amaral et al., 2016; Botelho et al., 2015). After validation, mechanistic classification according to the ERQC checkpoints and assessment of CFTR rescue additivity with the state-of-the art folding correctors, five kinase proteins were selected, and their MoA studied. Of these, G Protein-Coupled Receptor Kinase 5 (GRK5) was selected for detailed studies. GRK5 was found to rescue F508del-CFTR traffic and function upon inhibition with siRNAs or a recently reported selective inhibitor—(R,Z)-3-((4-(2-Bromoacetamido)-3,5-dimethyl-1H-pyrrol-2-yl)-methylene)-2-oxo-N-(1-phenylethyl)indoline-5-carboxamide—in a manner additive to CFTR correctors.

GRK5 is among those proposed as a potential new therapeutic target for CF since GRK5 inhibition may functionally release F508del-CFTR from the ER.

### BRIEF DESCRIPTION OF DRAWINGS

For easier understanding of this application, figures are attached in the annex that represent the preferred forms of implementation which nevertheless are not intended to limit the technique disclosed herein.

FIG. 1. Workflow of siRNA-based F508del-CFTR traffic screens. (A) The primary microscopy-based screen was based on screening the Ambion extended druggable genome siRNA library with a CFBE cell line expressing a mCherry-Flag-F508del-CFTR traffic reporter (Botelho et al., 2015). The library encompasses nearly half of the human genome (9128 genes, almost all of which targeted by 3 individual siRNAs. A subset of the siRNAs was discarded for targeting multiple or non-protein-coding genes (as of Ensembl 78), or due to damages in the assay plates. The primary screen highlighted 228 hit siRNAs—targeting 227 genes—which produced significant increases in F508del-CFTR delivery to the PM. Most of the primary screen hit genes (225 genes) were re-screened with additional siRNAs (2 siRNAs/gene). A total of 35 genes, targeted by 70 siRNAs, were confirmed as hits. For complementary validation studies a library of siRNAs targeting the 35 confirmed hit genes, as well as on two additional kinases (DGKG, GRK5) which were part of the primary screen hits. Significant increases in F508del-CFTR glycosylation (Western blot) or function (hsYFP quenching) were achieved by knocking down 36 or 21 of these genes. The overlap between YFP and WB is 20 genes and 25 siRNAs. Downstream studies focused on a panel of 5 kinase genes, selected for their druggability. In parallel, reporter cell lines expressing wt-CFTR, the DD/AA-CFTR mutant or genetic F508del-CFTR revertants affecting known ERQC checkpoints were used to screen the confirmation siRNA library, thereby allowing to classify the hit genes according to their dependence on different ERQC mechanisms. (B) Waterfall plot with Z-score5×5 for PM F508del-CFTR (median±SEM) obtained with all siRNAs analyzed in the primary screen and (C) inset showing the subset of 228 primary siRNAs which significantly increase PM F508del-CFTR. Dotted lines sow the thresholds for enhancer (Z-score5×5>+1) or inhibitor siRNA hits (Z-score5×5<−1). For most siRNAs n=3.

FIG. 2. Overview of the confirmation siRNA screen. (A) Waterfall plot displaying the ranked modified Z-scores describing PM localization of F508del-CFTR as a function of treatment with all siRNAs in the primary screen. Values are median±SEM. (B) Representative microscopy data from the confirmation screen hit genes. Microscopy images of controls and selected hit genes. Negl is a non-targeting siRNA. VX-809 (3 μM) was added to selected wells containing Negl to obtain a positive control for the screen. The loss of CFTR expression (mCherry) in CFTR siRNA-treated cells reports on a high transfection efficiency. Gene symbols and respective siRNAs catalogue numbers are shown. Scale bar: 50 μm. (C) Z-score for the PM and traffic efficiency quantification, shown as median±SEM. For most siRNAs n=3.

FIG. 3. Effect of hit siRNAs on F508del-CFTR processing assessed by WB. (A) CFBE cells overexpressing F508del-CFTR were transfected with siRNAs targeting 34 of the 35 confirmed hit genes (LDLRAD could not be included) and analyzed by WB for processing to confirm effects on traffic rescue. Wt-CFTR was included for reference. (A) CFTR detection in a WB membrane. wt-CFTR was included for reference. The immature, core glycosylated (band B) and processed, fully glycosylated (band C) forms of CFTR are shown. (B) Quantification of band C, normalized to the corresponding loading controls. ‘*’ indicates statistical significance from Negl-treated cells (p<0.05, one-way ANOVA followed by Dunnett's post-hoc test, n=3). 58 out of the 68 tested siRNAs showed significant band C rescue versus the control. Plot values are mean±SD.

FIG. 4. Measurements of HS-YFP fluorescence quenching for hit siRNAs. Plots show data on siRNAs which produced quenching rate enhancements above +1 Z-score units on cells expressing F508del-CFTR (*). For some genes, quenching rate enhancements were observed for 2 distinct siRNAs. The corresponding values for wt-CFTR are also indicated. Values are average of 4 assays for most siRNAs. VX-809 and VX-661 were assayed at 3 and 5 μM, respectively.

FIG. 5. Hierarchical clustering of the classification screen results. (A) Number of common hit genes for the several CFTR variants. (B) Simplified representation of the first panel, showing only hit kinases. (C) Hierarchical clustering of a subset of the classification screen results. The plot shows data for the 35 siRNAs which enhance F508del-CFTR PM localization. The x-axis represents CFTR variants and each line in the y-axis is a single siRNA. Values are the Z-score for PM localization. Data was clustered in the x and y axes, as shown by dendrograms. The rows dendrogram highlights the 7 clusters mentioned in the results description. (D) Co-occurrence of genes in the F508del-CFTR traffic screens (this study) and other CFTR-related datasets: F508del-CFTR interactome in HBE41o- cells (Pankow et al., 2015), F508del-CFTR interactome in CFBE 41o- cells (Canato et al., 2018; Reilly et al., 2017), genes whose silencing significantly rescued F508del-CFTR activity (Tomati et al., 2018), candidate modifier genes of CF lung disease (Dang et al., 2020), ENaC activating genes in A549 cells (Almaça et al., 2013) and protein secretion machinery in HeLa cells (Simpson et al., 2012). The outer track contains one tick mark for each gene in the dataset (gray). Inter-dataset comparisons were performed after converting the reported gene identifiers to Uniprot IDs (see Methods). During the conversion process, genes for which an updated Uniprot ID could not be found were discarded, explaining the discrepancy in the gene counts in the original publications and this figure. For this study, the gene tick marks represent the Z-score. the outer track represents the primary screen hits. The subset of 35 confirmed genes is represented in the inner track. Common genes are linked with arcs in the center of the plot. Common genes in published datasets are linked by thin gray arcs. Primary screen hits are linked by thin dark red arcs. Confirmation screen hits are kinked by thin bright red arcs. Hit kinase genes are linked by thick bright red arcs. The symbols of hit kinases as well as primary and confirmation screen hit genes are also written in red. The only instance where one of the hit kinases is reported in another study is GRK5, which is an ENaC activating gene.

FIG. 6. Additivity of hit kinase KD with CFTR correctors on F508del-CFTR processing rescue. CFBE cells stably expressing F508del-CFTR were transfected with Negl or siRNAs targeting STYK1, TPK1, DGKG, GRK5 or LRRK1 and incubated for 24 h with DMSO (vehicle), 3 μM VX-809 (A,B), 5 μM VX-661 (C,D) or VX-661 combined with 3 μM VX-445 (E,F). Representative WB membranes (A, C, E) and densitometric quantification of CFTR and C normalized to the calnexin loading control (B, D, F). ‘#’ indicates statistical significance from the corresponding DMSO control (p<0.05 in double-tailed unpaired t-test, n=3). Plot values are mean±SD.

FIG. 7. Quantification of the knock-down efficiency of hit kinases. CFBE cells stably expressing F508del-CFTR were treated with siRNAs targeting the 5 hit kinases, as performed for other figures, omitting the incubation of CFTR correctors. The abundance of the mRNA coding for each gene is shown, normalized for cells treated with the non-targeting Negl siRNA (mean±SD, n=3).

FIG. 8. Scheme of (R,Z)-3-((4-(2-Bromoacetamido)-3,5-dimethyl-1H-pyrrol-2-yl)-methylene)-2-oxo-N-(1-phenylethyl)indoline-5-carboxamide synthesis. i) (S)-1-phenylethan-1-amine, HATU, DIPEA, DMF, rt, overnight, 84%; ii) 3,5-dimethyl-4-nitro-1H-pyrrole-2-carbaldehyde, piperidine, EtOH, 95° C., 2 h, 87%; iii) zinc powder, AcOH, EtOAc, EtOH, 50 C, 2 h, 79%; iv) bromoacetic acid, HATU, DIPEA, DMF, rt, overnight, 55%.

FIG. 9. GRK5 inhibition with 9 g rescues F508del-CFTR traffic and function. (A) CFBE cells stably expressing F508del-CFTR were incubated with increasing doses of (R,Z)-3-((4-(2-Bromoacetamido)-3,5-dimethyl-1H-pyrrol-2-yl)-methylene)-2-oxo-N-(1-phenylethyl)indoline-5-carboxamide (9 g) or VX-661 3 μM and analyzed with Western blot. Densitometric quantification of band C (B) and CFTR processing [C/(B+C)](C). Statistically significant band C rescue is observed at 0.3 and 1.0 μM of (R,Z)-3-((4-(2-Bromoacetamido)-3,5-dimethyl-1H-pyrrol-2-yl)-methylene)-2-oxo-N-(1-phenylethyl)indoline-5-carboxamide (9 g). Processing is not significantly changed due to proportional increases in band B. Symbols indicate statistical significance from DMSO (“*”) or VX-661 (“#”) lanes (p<0.05, one-way ANOVA followed by Dunnett's post-hoc test, n=3). Plot values are mean±SD. (D) Additivity of (R, Z)-3-((4-(2-Bromoacetamido)-3,5-dimethyl-1H-pyrrol-2-yl)-methylene)-2-oxo-N-(1-phenylethyl)indoline-5-carboxamide (9 g) (1 μM) to CFTR correctors VX-661 (5 μM), VX-445 (3 μM) and VX-661+VX-445 and densitometric quantification of band C (E) and B (F). (R,Z)-3-((4-(2-Bromoacetamido)-3,5-dimethyl-1H-pyrrol-2-yl)-methylene)-2-oxo-N-(1-phenylethyl)indoline-5-carboxamide (9 g) is additive to all corrector treatments regarding bands C and B (#p<0.05 unpaired t-test, n=4). (G) halide-sensitive YFP quenching of CFBE cells expressing F508del-CFTR and incubated with (R,Z)-3-((4-(2-Bromoacetamido)-3,5-dimethyl-1H-pyrrol-2-yl)-methylene)-2-oxo-N-(1-phenylethyl)indoline-5-carboxamide (9 g) (1 μM) and/or VX-661 (5 μM), VX-445 (3 μM) and VX-661+VX-445. Iodide was added at the indicated time point to initiate quenching. (H) Quenching rate at the moment of iodide addition. (R,Z)-3-((4-(2-Bromoacetamido)-3,5-dimethyl-1H-pyrrol-2-yl)-methylene)-2-oxo-N-(1-phenylethyl)indoline-5-carboxamide (9 g) is additive to all corrector treatments regarding F508del-CFTR functional rescue (#p<0.05 double-tailed unpaired t-test, n=4). Plot values are mean±SD.

FIG. 10. Model for F508del-CFTR rescue via GRK5 inhibition. GRK5 is part of the machinery which activates CFTR endogenously, in response to β2-adrenergic receptor (β2AR) ligands such as epinephrine, isoprenaline or albuterol. Ligand binding is transduced intracellularly by the hetero-trimeric G proteins associated with β2AR, which activate adenylyl cyclase, the enzyme which converts ATP to cAMP. The cAMP pool activates CFTR by two distinct mechanisms: (i) activation of protein kinase A (PKA), which phosphorylates CFTR's R domain in an ATP-dependent manner as the first step in the gating cycle which opens the channel, and (ii) EPAC1-dependent stabilization of CFTR at the PM. Adenylyl cyclase can be artificially activated with forskolin (Fsk). The cAMP pool is depleted by phosphodiesterases (PDE), which can be inhibited with 3-Isobutyl-1-methylxanthine (IBMX). GRK5 phosphorylates activated β2AR, which then becomes substrate for R-arrestin and is endocytosed to interrupt receptor signaling. Endocytic vesicles are recycled to the PM to restore cell sensitization. GRK5 kinase activity is promoted by Ca2+-calmodulin (CaM). GRK5 inhibition with siRNAs or (R,Z)-3-((4-(2-Bromoacetamido)-3,5-dimethyl-1H-pyrrol-2-yl)-methylene)-2-oxo-N-(1-phenylethyl)indoline-5-carboxamide (9 g) releases F508del-CFTR from the ER, increases the steady state of CFTR at the PM and yields higher CFTR-dependent Cl⁻ conductance. The link between GRK5 and F508del-CFTR ER release is not completely clear but given that GRK5 is not a CFTR interactor and its inhibition is additive to VX-661 a direct GRK5-CFTR mechanism is unlikely. Since GRK5 is expressed ubiquitously, this pathway may be active on several cell types which express CFTR. Adapted from (Boccella et al., 2019; Traynham et al., 2016).

### DETAILED DESCRIPTION OF EMBODIMENTS

Now, preferred embodiments of the present application will be described in detail with reference to the annexed drawings. However, they are not intended to limit the scope of the invention.

The present invention relates to a method of identifying agents for the treatment of Cystic Fibrosis caused by the mutation F508del in the gene encoding the CFTR protein (F508del-CFTR), wherein a candidate agent contacts with at least one gene sequence selected from SEQ ID NOs: 228 to 478 or with at least one of its protein products of Seq.ID: 1 to 227 as therapeutic targets, and the ability of the candidate agent to modulate the activity of the F508del-CFTR protein and its plasma membrane traffic is determined.

The gene sequences SEQ ID NOs: 228 to 478 were identified by a high-throughput siRNA cell-based microscopy screen aimed at identifying gene KDs which enhance F508del-CFTR plasma membrane levels, as potential candidates for drug therapy in the treatment of Cystic Fibrosis. Of those, Seq.ID: 232, 237, 238, 250, 253, 257, 259, 263, 276, 285, 296, 297, 301, 317, 318, 319, 333, 338, 369, 378, 396, 401, 405, 409, 410, 411, 417, 420, 421, 430, 442, 450, 458, 459, 471, 472 and 478 were identified as the top hits for which KD enhances the PM localization of F508del-CFTR.

Five kinases (TPK1, LRRK1, STYK1, GRK5, DGKG, Seq. ID: 237, 238, 232, 396, 233 and 248 respectively), were identified as showing F508del-CFTR rescue in the microscopy or Western-Blot assays, and are now disclosed as potential drug targets for F508del-CFTR CF.

Activity modulation is affected by inhibiting gene expression of at least one gene sequence selected from Seq.ID: 228 to 478 or inhibiting their protein product's activity. Inhibition can be affected by reducing gene expression of at least one gene sequence selected from SEQ ID NOs: 228 to 478 or be affected by inhibition on the protein level.

The candidate agent must be suitable to inhibit the activity of at least one gene sequence selected from Seq.ID: 228 to 478 or their protein product by more than 20% relative to the activity in the absence of the candidate agent.

The capability of the candidate agent to bind to a protein product, or a domain thereof, from at least one gene sequence selected from SEQ ID NOs: 228 to 478 is determined.

In one embodiment (R,Z)-3-((4-(2-Bromoacetamido)-3,5-dimethyl-1H-pyrrol-2-yl)-methylene)-2-oxo-N-(1-phenylethyl)indoline-5-carboxamide is the candidate agent for the treatment of Cystic Fibrosis caused by the mutation F508del in the gene encoding the CFTR protein.

The candidate agent identified by the method herein disclosed is thus suitable for the treatment the treatment of Cystic Fibrosis caused by the mutation F508del in the gene encoding the CFTR protein.

Also disclosed in a pharmaceutical composition comprising an agent suitable to modulate F508del-CFTR activity and its plasma membrane traffic through at least one gene sequence selected from SEQ ID NOs: 228 to 478 or through their protein product as therapeutic targets.

In one embodiment, the pharmaceutical composition can further comprise at least one pharmaceutically acceptable diluent, carrier, adjuvant or excipient.

Cystic Fibrosis caused by the mutation F508del in the gene encoding the CFTR protein can be treated by using an effective amount of an agent that modulates the activity of F508del-CFTR protein and its plasma membrane traffic through at least one gene sequence selected from SEQ ID NOs: 228 to 478 or through their protein product as therapeutic targets.

### Experimental Data

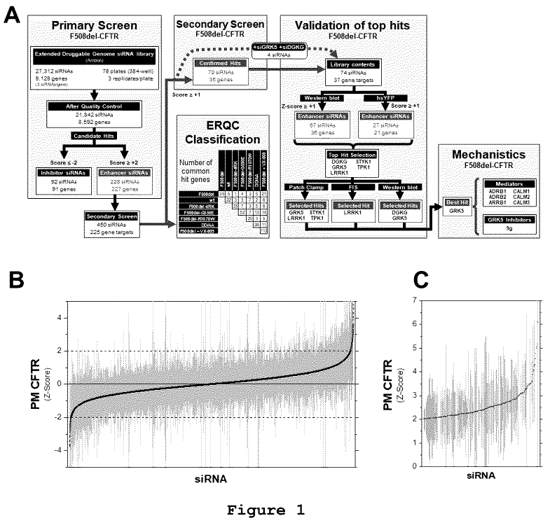

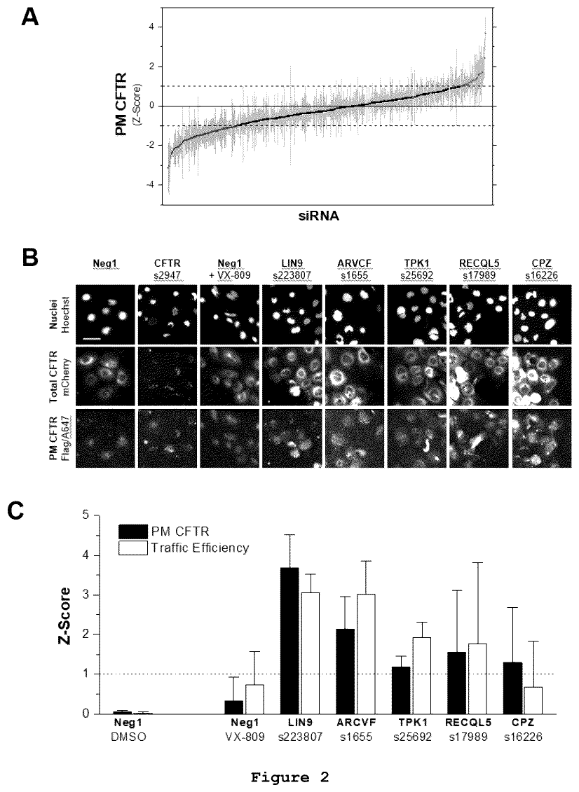

In this application it is described a high-throughput siRNA cell-based microscopy screen aimed at identifying gene KDs which enhance F508del-CFTR PM levels. Screening a commercially available siRNA library targeting nearly half of the human genome, selecting hit genes (FIG. 1) and re-screening the hits with different siRNAs confirmed 35 genes (FIG. 2) whose KD enhances the PM localization of F508del-CFTR, which were termed top hits. This stringent criterion equates to a 15% hit validation rate, somewhat lower than the 30% usually reported (Almaça et al., 2013; Neumann et al., 2010; Simpson et al., 2012). However, this can be explained by several reasons (low siRNA efficiency/specificity) and does not necessarily imply that for hits identified in the primary screen which were not confirmed in secondary screen a role on F508del-CFTR traffic is excluded.

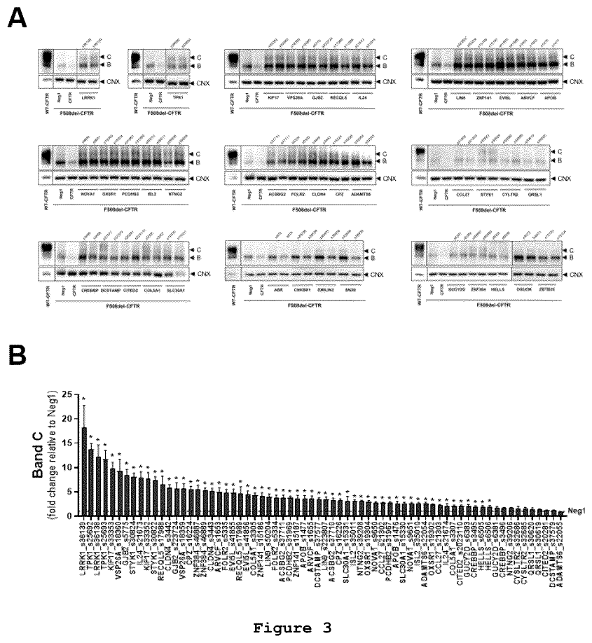

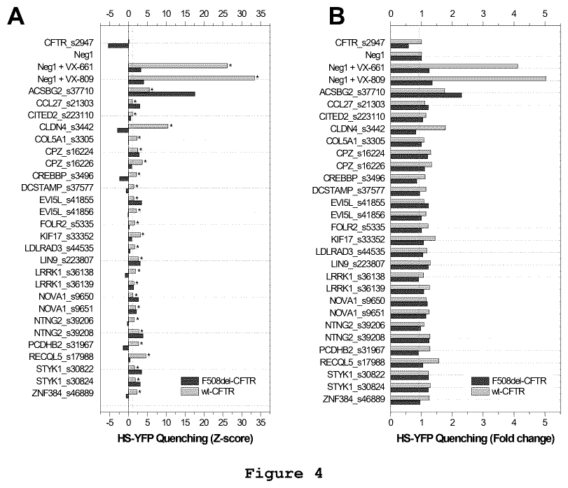

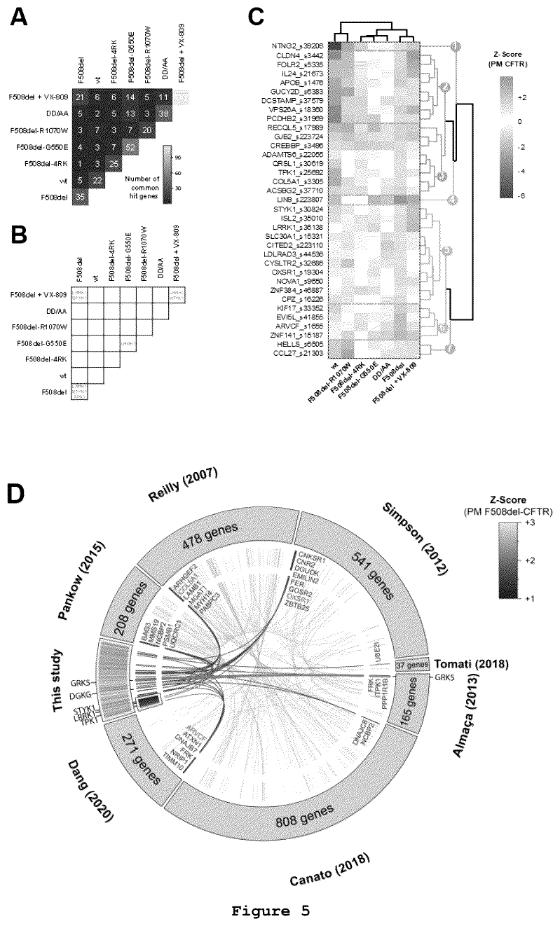

To ascertain the validity of these top hit genes as novel therapeutic targets for CF gene KDs were performed while assessing F508del-CFTR processing by WB (FIG. 3), as well as functional rescue of CFTR-mediated anion conductance using the HS-YFP fluorescence quenching assay (FIG. 4). It was found that KD of 21 of these top hits also rescued F508del-CFTR function. The WB-based validation pinpointed 50 hit KDs (32 of the 35 confirmed genes and 18 additional genes targeted by sub-optimally scoring siRNAs in the secondary screen) as significantly rescuing F508del-CFTR band C, the fully glycosylated CFTR form characteristically found in the PM (FIG. 3). Expression of most of these genes in lung tissue was confirmed with RT-PCR. CFTR variants which are sensitive or resistant to ERQC checkpoints had mixed effects regarding CFTR rescue additivity when hit gene KDs were performed (FIG. 5 A-C).

We noticed that among the 50 gene KDs which shown F508del-CFTR rescue in the microscopy or WB assays there were 5 kinases (TPK1, LRRK1, STYK1, GRK5, DGKG), which were selected for additional validation not only because of their amenability to pharmacological modulation but because their KD was among the conditions generating the highest F508del-CFTR band C rescue. Additionally, by analyzing a selected set of CF or CFTR-related gene datasets (FIG. 5D) it was surmised that the kinases could be novel CFTR regulators given that there was only one report of one of the kinases in the scanned datasets: GRK5 KD was also found to inhibit ENaC. This phenotype is desirable in the clinic because the loss of Cl⁻ secretion via CFTR due to CF-causing mutations is accompanied by Na⁺ hyperabsorption via ENaC, which exacerbates airway dehydration.

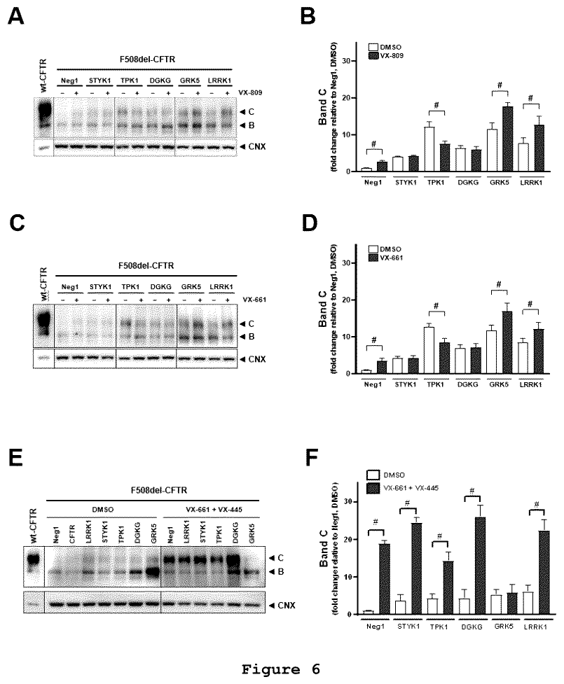

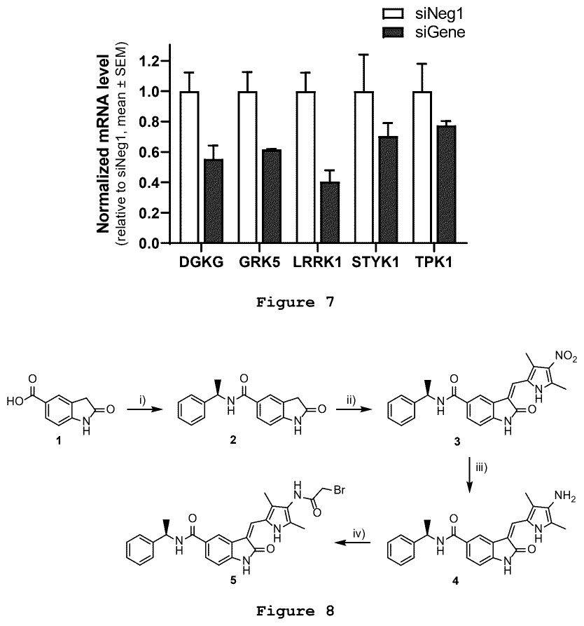

To ascertain the potential biomedical relevance of inhibiting these kinases to achieve F508del-CFTR rescue WB and patch clamp assays were performed in combination with treatment with clinical CFTR correctors: VX-809, VX-661 and VX-661 plus VX-445. In WB all KDs were additive with at least 1 corrector treatment, suggesting an independent MoA (FIG. 6). It was also noted that some of the kinase KDs (e.g. GRK5) could share the MoA of VX-445, due to deleterious effects on combining the KD with corrector treatment. RT-PCR analysis revealed that in these experiments mRNA levels for the 5 kinases were reduced between 22 and 60% (FIG. 7). The hit kinases were not hits in similar siRNA-based screens examining F508del-CFTR functional rescue when targeting the druggable genome (Tomati et al., 2018) or kinome (Perkins et al., 2018) (FIG. 5D).

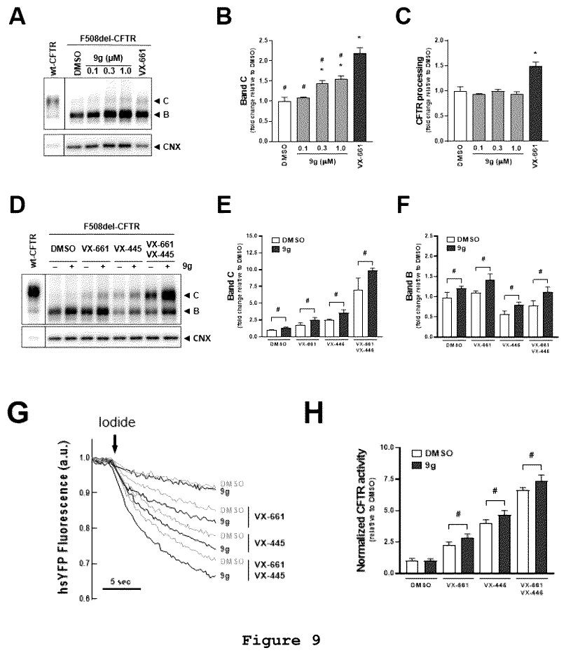

Altogether, the data pointed to GRK5 as a therapeutically relevant potential new F508del-CFTR regulator gene, not only for the relevant rescue when knocking down the gene, but also for the indications on its MoA. To validate GRK5 as a potential novel therapeutic target for CF the enzyme was inhibited with a recently published selective and covalent inhibitor: (R,Z)-3-((4-(2-Bromoacetamido)-3,5-dimethyl-1H-pyrrol-2-yl)-methylene)-2-oxo-N-(1-phenylethyl)indoline-5-carboxamide (Rowlands et al., 2021, FIG. 8). Low micromolar concentrations of this compound achieved significant F508del-CFTR processing and function rescue, which was additive to VX-809, VX-445 and VX-661 (FIG. 9), suggesting a potential added benefit.

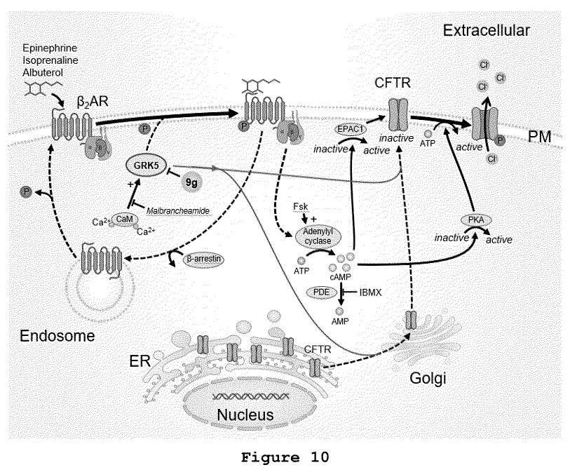

It was hypothesized that the GRK5 inhibition could yield F508del-CFTR rescue through the well-established pathway of endogenous CFTR activation through the beta2-adrenergic receptor (β2AR) (Boccella et al., 2019; Traynham et al., 2016) (FIG. 10). β2AR is a G-protein coupled receptor and binding of extracellular β2AR agonists (epinephrine, amlexanox) is transduced intracellularly through trimeric G proteins, which activate adenylyl cyclase, the enzyme which converts ATP into cyclic AMP (cAMP). cAMP contributes to functionally activate CFTR through two independent pathways: activation of protein kinase A (PKA) which phosphorylates CFTR's R domain as the first step in channel gating; and EPAC1-dependent stabilization at the PM. Adenylyl cyclase can be artificially activated with forskolin and the cAMP pool can be depleted by phosphodiesterases (PDE), which are inhibited by IBMX. GRK5 is recruited by the G proteins to phosphorylate β2AR. This determines β2AR endocytosis through β-arrestins, which terminates the signal. Eventually, β2AR is recycled to the PM to restore cell sensitization. Endogenously, GRK5 is activated by interaction with Ca²⁺-calmodulin (CaM), a process which can be inhibited with malbrancheamide. It is not clear how GRK5 inhibition releases F508del-CFTR from the ER, but available CFTR interactomes seem to rule out a direct GRK5-CFTR interaction. It is also not clear whether the possible impact on the cellular cAMP pool may be part of the MoA. The contribution of calmodulin was not assessed because that would be a target more upstream than GRK5.

GRK5 as well as the closely related GRK2 are ubiquitously expressed. Most preclinical research on these proteins relates to their role in cardiac hypertrophy associated to heart failure. Until recently, there were no specific GRK5 inhibitors. CCG215022 is a well-known pan GRK inhibitor with similar IC50 for GRK2, 5 and 1: 0.15±0.07 μM, 0.38±0.06 μM and 3.9±1 μM, respectively. It does not affect GRK1 or PKA but lacks overall specificity (Homan et al., 2015). (R, Z)-3-((4-(2-Bromoacetamido)-3,5-dimethyl-1H-pyrrol-2-yl)-methylene)-2-oxo-N-(1-phenylethyl)indoline-5-carboxamide is the first GRK5-specific inhibitor (IC50=8.6 nM and covalent binding) with 1400-fold selectivity against GRK2 (Rowlands et al., 2021). Out data suggests that (R,Z)-3-((4-(2-Bromoacetamido)-3,5-dimethyl-1H-pyrrol-2-yl)-methylene)-2-oxo-N-(1-phenylethyl)indoline-5-carboxamide could hold therapeutic relevance in CF, by rescuing F508del-CFTR in a manner additive to current folding correctors. The low micromolar range where F508del-CFTR rescue was observed on cellular models is comparable to the working concentration of CFTR clinical correctors on the same systems, suggesting a relevant potency. Finally, the work suggests that the preclinical relevance of (R,Z)-3-((4-(2-Bromoacetamido)-3,5-dimethyl-1H-pyrrol-2-yl)-methylene)-2-oxo-N-(1-phenylethyl)indoline-5-carboxamide should be evaluated in cellular and animal models with the goal of establishing an improved CF therapy in combination with current clinical drugs.

The results point to GRK5 as a putative novel therapeutic target for Cystic Fibrosis, which can be inhibited with the selective inhibitor (R,Z)-3-((4-(2-Bromoacetamido)-3,5-dimethyl-1H-pyrrol-2-yl)-methylene)-2-oxo-N-(1-phenylethyl)indoline-5-carboxamide. This compound holds the potential to become a new and improved CF therapy, to be combined with current CFTR-targeting clinical drugs.

### Examples

### 1. Methods

### CFTR Constructs and Cell Lines

Cell transfection was performed with a reverse transfection protocol (Erfle et al., 2007), whereby assay plates are coated with a lyophilized mixture of siRNA plus transfection reagent before cells are seeded. Contact time with the siRNA—i.e. transfection time—was 72 h. Expression of the traffic reporter was induced with doxycycline during the last 48 h of the assay. This setup, where the traffic reporter is expressed in a cellular milieu depleted for the siRNA target gene was selected so as to increase the assay sensitivity for factors regulating CFTR biogenesis. Imaging was performed with a widefield fluorescence microscope, which allows detecting all cellular fluorescence in a single image. The library was laid out on microscopy-grade 384 well plates, where each well contained only one siRNA molecule. The coating procedure failed in 8 of the plates, which could not be used for screening. The screening data was subjected to a quality control process which filtered out the following cases: (i) siRNAs which were suspect of targeting no protein-coding genes or simultaneously targeting multiple targets (see methods); (ii) cells which did not express significant CFTR amounts; (iii) out-of-focus images; and (iv) wells with a low cell count.

CFTR traffic reporters were built by fusing mCherry N-terminally to the CFTR molecule via a small linker (QISSSSFEFCSRRYRGPT) and by inserting a Flag tag sequence (DYKDDDDK) in the fourth extracellular loop of CFTR, as described elsewhere (Almaça et al., 2011; Botelho et al., 2015). CFBE cells (CFBE41o-) were stably transduced with lentivirus encoding the mCherry-Flag-CFTR constructs under the control of a Tet-ON promoter, thereby generating traffic reporter cell lines. Traffic reporters were generated for several CFTR variants: wt- and F508del-CFTR (reported in (Botelho et al., 2015)), variants containing F508del revertant mutations F508del-4RK (R29K, R516K, R555K, R766K), F508del-R1070W-CFTR, F508del-G550E-CFTR, as well as the DD/AA-CFTR mutant (Canato et al., 2018). Reporter cell lines were grown in Dulbecco's Modified Eagle's Medium (DMEM) containing 4.5 g/L Glucose and L-Glutamine (Lonza, #12-604F) supplemented with 10% (v/v) foetal bovine serum (FBS, Gibco #10106), 2 μg/ml puromycin (Sigma, #P8833) and 10 μg/ml Blasticidin (InvivoGen, #ant-bl) at 37° C. in 5% CO₂.

CFBE41o- cells constitutively co-expressing the HS-yellow fluorescent protein (HS-YFP) YFP-H148Q/I152L as well as wt- or F508del-CFTR (described in (Sondo et al., 2011)) were a kind gift from Dr Nicoletta Pedemonte (IRCCS Istituto G. Gaslini, Genoa, Italy). These cells were cultured in Minimum Essential Medium (MEM) supplemented with 10% FBS, 2 mM L-glutamine, puromycin 2 μg/ml and G418 200 μM (Sigma, #A1720) at 37° C. in 5% CO₂.

CFBE41o- cells constitutively expressing wt- or F508del-CFTR were grown in DMEM supplemented with 10% FBS, at 37° C. in 5% CO₂.

### Chemistry General Remarks

All solvents were purchased from commercial sources and were dried according to standard methods. All chemicals and starting materials (2-oxoindoline-5-carboxylic acid, (S)-1-phenylethan-1-amine, HATU, 3,5-dimethyl-4-nitro-1H-pyrrole-2-carbaldehyde, bromoacetic acid, DIPEA, piperidine and zinc powder) were purchased from Fluorochem and Sigma-Aldrich. Reactions were monitored by TLC using 0.25 mm silica gel 60 F254 TLC plates purchased from Merck. Spots were visualized under ultraviolet light (254 nm).

1H, 13C APT NMR spectra were acquired on a Bruker Avance 400 spectrometer equipped with a 5 mm QNP probe operating at 400.16 MHz and 100.61 MHz for 1H and 13C respectively, at 293K. Spectra of all compounds were recorded using DMSO-d6 (99.9%; EurIsotop, UK). Spectroscopic data of all compounds was matched with the one previously reported (Rowlands et al., 2021). High-Resolution-mass spectra (HR-MS) and low-resolution mass spectra (MS) spectra were performed on an Impact II QTOF (Bruker, Bremen, Germany) mass spectrometer with an electrospray ionization source (ESI). The method consisted of direct infusions with MS/MS scans, in the positive and negative modes

### Synthetic Procedure

(R,Z)-3-((4-(2-Bromoacetamido)-3,5-dimethyl-1H-pyrrol-2-yl)-methylene)-2-oxo-N-(1-phenylethyl)indoline-5-carboxamide (5) was synthesized employing slight modifications of the Rowlands et al. method (Rowlands et al., 2021). In the second step, reaction time was cut in half and the yield increased to 87%. In the last step of the synthesis, the reported coupling agent was DMTMM with a yield of 39%, while HATU was used which afforded the final compound (5) in 55% yield (FIG. 8).

### CFTR Traffic Assay: Primary Screen

To establish an assay compatible with siRNA-based gene KD and small molecule treatments, a total experiment time of 72 h was selected, a consensus time in other studies (Dalemans et al., 1991; Pedemonte et al., 2011). In the primary screen CFTR intracellular localization was assessed in CFBE cells expressing the inducible mCherry-Flag-F508del-CFTR traffic reporter and subjected to siRNA KDs. To maximize transfection efficiency, cells were split 24 h before the start of the experiment to synchronize them in exponential growing phase. The experiment was initiated by seeding cells in microscopy-grade multi-well plates (384 wells) where each well was coated with a lyophilized mixture of an individual siRNA and a transfection reagent (see siRNA libraries below). Each well was seeded with 2,500 cells using a peristaltic pump (ThermoFisher Multidrop™ Combi). Antibiotics were removed at this stage. Expression of the mCherry-Flag-F508del-CFTR construct was induced with 1 μg/ml doxycycline (Dox, Sigma, #9891) starting at 24 h after seeding and continuing for 48 h, to allow for the 72 h siRNA contact time described above. This induction period allowed for sufficient construct expression, as measured by mCherry fluorescence. To obtain a positive control, wells containing a non-targeting siRNA (Scrambled) were induced in the presence of 3 μM VX-809 (Selleckchem #S1565) and a lower amount of FBS in the culture medium (0.1%).

After the 72 h assay time, cells undergo an immunofluorescence protocol to detect extracellular Flag tags in non-permeabilized cells, as described in (Amaral et al., 2016; Botelho et al., 2015). Briefly, cells are washed once in ice cold PBS supplemented with 0.7 mM CaCl₂ and 1.1 mM MgCl₂ (PBS⁺⁺), incubated 1 h with an anti-Flag antibody (1:500, Sigma #F1804), washed three times with ice cold PBS⁺⁺, incubated 20 min with 3% paraformaldehyde (PFA), washed three times with room temperature PBS⁺⁺, incubated 1 h with an anti-mouse Alexa Fluor 647 conjugated secondary antibody (1:500, Life Technologies #A31571), washed three times with PBS⁺⁺, incubated with Hoechst 33342 (200 ng/ml, Sigma #B2261) for 1 to 6 hours, washed with PBS⁺⁺ and stored at 4° C. until imaging. Antibodies are solubilized in PBS⁺⁺ containing 1% bovine serum albumin (BSA, Aldrich #A9647). PFA and Hoechst are solubilized in PBS⁺⁺.

### CFTR Traffic Assay: Secondary Screen

To confirm the siRNA hits found to significantly enhance F508del-CFTR traffic in the primary screen (primary screen hits), a secondary screen was performed. The 227 primary screen hit genes were re-screened with 2 additional siRNAs using the traffic assay described for the primary screen and CFBE cells expressing the mCherry-Flag-F508del-CFTR reporter. The siRNAs were selected so as to have improved stability and specificity (Silencer Select siRNAs, Ambion) and different sequences than the ones in the primary screen.

### CFTR Traffic Assay: ERQC Checkpoints Classification Screen

Classification of the primary screen hits in terms of their effect on the ERQC checkpoints which retain F508del-CFTR was pursued by re-screening the primary screen hits using the reporter cell lines expressing wt-CFTR, F508del-CFTR genetic revertants (4RK, G550E or R1070W) as well as the DD/AA variant.

siRNA Libraries

The CFTR traffic assays used Ambion siRNAs and transfection reagents. For the primary screen Ambion's Silencer Human Extended Druggable Silencer siRNA library was used. All siRNA sequences from this library were blasted against the transcriptomic data in Ensembl version 78, to reassign the gene target of each siRNA. Only siRNA molecules targeting a single protein-coding gene were considered for screening. The hits from the primary screen were re-screened with a cherry-picked library composed of Ambion Silencer Select siRNAs (2 siRNAs per gene target). Lipofectamine 2000 was used as a transfection reagent. In both cases, the microscopy plates were coated with a siRNA plus transfection reagent mixture and lyophilized (Botelho et al., 2015). At this point, plates were stored at room temperature in airtight boxes containing desiccating pearls. For the traffic assay, transfection was triggered by cell seeding (reverse transfection, as described in (Erfle et al., 2007)). At the moment of CFTR induction the siRNA target gene is—at least partially—absent.

Validation assays (WB and HS-YFP quenching) were performed with a cherry-picked siRNA library of Ambion Silencer Select siRNAs, where each of the 35 confirmed hits were targeted by the same 2 siRNAs used in the confirmation step.

### Image Acquisition

Automated cell imaging was performed at room temperature with a Leica DMI6000 B inverted epifluorescence microscope equipped with a metal halide light source (EL6000), a 10× NA 0.40 HC PL APO objective, a Hamamatsu Orca-Flash4.0 CMOS camera and filter cubes suitable for imaging the fluorophores used in the traffic assay: Hoechst 33342 (model A, Leica Microsystems: excitation: BP 340-380; dichroic mirror 400; emission filter LP 425), mCherry (model N2.1, Leica Microsystems: excitation BP 515-560; dichroic mirror 580; emission LP 590) and Alexa Fluor 647 (custom: excitation: BP 630-660; emission LP 670). Image acquisition was performed with the microscope being controlled by the Leica MatrixScreener software, which managed the software autofocus, multiposition imaging of each well (4 subpositions per well) and imaging of the three fluorophores. Images were stored as 16 bit OME TIF files.

### Image Analysis

Image quantification was performed with CellProfiler (Carpenter et al., 2006) using an analysis pipeline which performed background subtraction, cell segmentation, fluorescence integration and basic quality control (exclusion of cells undergoing mitosis, apoptosis or containing a significant number of saturated pixels). Background subtraction was performed using a dark field/flat frame algorithm using experimental (secondary and classification screens) or simulated (primary screen) correction images. Cell phenotype was assessed from three image features, extracted from each cell: the integrated mCherry fluorescence signal (which reports on total CFTR expression), the integrated Alexa Fluor 647 fluorescence signal (which reports on the amount of CFTR molecules inserted into the PM) and the ratio of the integrated fluorescence signals from Alexa Fluor 647 and mCherry (A647/mCherry, which reports on CFTR traffic efficiency). Screen hits were defined based on the quantification of the PM signal. The confirmation and classification library plates contained 5-8 wells with an siRNA targeting CFTR, as a transfection control. Only the plates where mCherry fluorescence was lost (i.e. abrogation of CFTR expression) in the majority of cells were scored.

### Data Analysis—Primary Screen

To score the CFTR traffic phenotype generated by each siRNA a statistical analysis algorithm was devised which takes into consideration the experimental context where the data was originally produced: i) multi-well plates (384 well); ii) each well corresponds to one siRNA treatment; iii) the same siRNA may occur at one or more wells in the same plate; iv) some wells correspond to controls (e.g. positive, negative or transfection controls); v) each well is imaged at 4 non-overlapping positions; vi) each imaging field encompasses multiple cells; vii) there are fluorescence measurements for each cell; viii) there are independent biological replicate experiments for each plate.

The statistical analysis algorithm takes as input image-based features (e.g. metadata, focus score, background fluorescence) and cell-based features (e.g. the image they belong to, metadata, fluorescence intensities, traffic efficiency ratio) generated by image analysis with CellProfiler and computes a modified Z-score for the PM fluorescence intensity and traffic efficiency ratio produced by each siRNA. The algorithm has been implemented in the R programming language and has the following steps: i) Import image and cell features; ii) Quality Control: exclude all data from unapproved plates (bad sample preparation), wells (wells with less than 100 cells), images (out-of-focus or high background images) and cells (aberrant morphology or fluorescence saturation); iii) Well Summary: take the PM fluorescence intensity and traffic efficiency values for all cells in a well and compute their respective medians; iv) Normalization: convert each well summary value into a score. Due to the lack of reliable non-targeting siRNAs in the primary screen library, the negative control measurements for each well were provided by neighbouring ones, as defined by a 5×5 matrix centered at the target well. For external wells, which do not have 24 neighbours, missing values were dropped. Therefore, primary screen data was normalized with a modified Z-score defined by:

$$Modified Z - score = \frac{x - Median ( {Neighbors}_{5 \times 5 , {plate}_{i}} )}{sd ( {Neighbors}_{5 \times 5 , {plate}_{i}} )}$$

where x is the well summary value, sd is the standard deviation and Neighbors_{5×5, plate}_{ᵢ} is the ensemble of negative control values; v) Plate summary: compute the median of the normalized PM staining and traffic efficiency values for all wells which have the same siRNA treatment in the same plate; vi) Treatment summary: take the plate summary values and compute their median across replicate plates. The median-summarized values are the ones reported herein.

### Data Analysis—Confirmation and Classification Screens

In the confirmation and classification siRNA libraries 10 to 18 wells in each 384 well plate contained a bona fide non-targeting siRNA (Negl), which was used as a negative control. For these results a standard Z-score was computed through the following algorithm (Botelho et al., 2015): i) Quality Control: exclude all data from unapproved plates (low transfection efficiency in wells containing CFTR siRNA, bad sample preparation), wells (wells with less than 100 cells), images (out-of-focus or high background images) and cells (aberrant morphology or fluorescence saturation); ii) Well Summary: take the PM fluorescence intensity and traffic efficiency values for all cells in a well and compute their respective medians; iii) Normalization: convert each well summary value into a Z-score, using the average and standard deviation of wells containing the non-targeting Negl siRNA; iv) Plate summary: compute the median of the normalized PM staining and traffic efficiency values for all wells which have the same siRNA treatment in the same plate; v) Treatment summary: take the plate summary values and compute their median across replicate plates; vi) The median-summarized values are the ones reported herein.

### Measurements of Halide-Sensitive (HS-)YFP Fluorescence Quenching

This assay was used to assess whether the enhanced F508del-CFTR traffic recorded when the reporter cell line was transfected with confirmed hit siRNAs also corresponded to enhanced F508del-CFTR ionic transport function. As in the microscopy-based screens, a 72 h siRNA transfection time was selected. The assay followed a previously reported protocol (Sondo et al., 2011). CFBE cells co-expressing HS-YFP and wt- or F508del-CFTR were grown to confluency (37° C., 5% CO₂) and split to 50% confluency. On the following day, the experiment was initiated by seeding cells onto siRNA-coated 96 well plates using a peristaltic pump (ThermoFisher Multidrop™ Combi). Each well was seeded with 10,000 cells. Antibiotic-free medium was used. 48 h after seeding, positive controls were generated by adding the CFTR correctors VX-809 (3 μM) or VX-661 (5 μM, Selleckchem #S7059) to selected wells containing the non-targeting Negl siRNA. Serum concentration in these wells was reduced to 0.1%, 72 h after seeding, cells were washed two times with PBS (containing 137 mM NaCl, 2.7 mM KCl, 8.1 mM Na₂HPO₄, 1.5 mM KH₂PO₄, 1 mM CaCl₂, and 0.5 mM MgCl₂) and stimulated for 30 min with PBS containing Forskolin (20 μM, Sigma #F6886) and VX-770 (3 μM, Selleckchem #S1144) at 37° C. in the absence of CO₂. Each well was added 60 μl of the stimulation solution. After the stimulation period, each plate was transferred to a fluorescence plate reader equipped with fast reagent injectors (Tecan Infinite F200 Pro) and excitation (BP 475-495 nm) and emission (BP 523-458 nm) filters suitable for YFP fluorescence. Each assay consisted of a continuous 16 s fluorescence reading with 2 s before and 14 s after injection of 160 μl of a PBS where Cl⁻ had been replaced by I⁻ (final I⁻ concentration in the well: 100 mM). The pH of all PBS-containing solutions was rigorously set at pH 7.4 immediately before use to avoid artifacts in the fluorescence reading. The resulting kinetics was background subtracted and normalized to the pre-injection fluorescence value. To determine the fluorescence quenching rate associated with I⁻ influx, the final 14 s of the kinetics in each well were fitted with a single decay exponential curve and the derivative at the injection time (i.e. the quenching rate) was extracted from the curve parameters. To make the data comparable to the microscopy scoring the negative of the quenching rate was taken and converted it to a Z-score or to a fold change versus the negative control. This transformation has the advantage that positive Z-scores report on the KDs which activate F508del-CFTR above the negative control.

### Western Blotting (WB)

WB analyses were used as a readout for CFTR processing (and ER exit) to confirm the effect of gene KDs on CFTR PM levels, and to examine the additivity of gene KDs with modulation with folding corrector. The assay used CFBE cells constitutively expressing wt-, F508del-, F508del-G550E-, F508del-R1070W-, F508del-4RK-, DD/AA-, G85E- or N1303K-CFTR. Confluent cell cultures were split to 50% confluency, to stimulate cell proliferation. 24 h later, cells were seeded into 12-well plates which had been coated with siRNAs for reverse transfection (87,500 cells/well). Each plate included a non-targeting Negl siRNA as negative control and a CFTR targeting siRNA as transfection control. Cells were grown for 48 h in the presence of siRNAs. When required, VX-809 (3 μM), VX-661 (5 μM) or VX-445 (3 μM, in combination with VX-661) were added in the last 24 h of siRNA contact, along with matching DMSO controls.

WB samples were prepared by lysing transfected cells with Laemmli sample buffer. The buffer contained no bromophenol and was supplemented with a protease inhibitor cocktail (Roche, Complete: one tablet per ml), benzonase and magnesium chloride. Cells were scrapped manually at 4° C. and lysates were stored at −20° C. until analysis.

Following quantification, the same protein amount of each lysate was loaded on SDS-PAGE gels (7.5% or 10%), electrophoresed, blotted and probed with the following antibodies: anti-CFTR (CFFT 596, 1:1000-1:3000), or anti-Calnexin (1:3000) the latter as loading control. The membranes were developed with a luminescent enzymatic reaction. wt-CFTR samples were loaded at lower concentration in order to allow proper visualization of the much more abundant CFTR bands.

Images were quantified with Bio-Rad's Image Lab software, with local background subtraction. The quantification of each band was transformed to the fold change versus the loading control band. To compare across bands, results were reported as fold change versus F508del-CFTR-expressing cells transfected with the Negl siRNA in the absence of correctors.

### Patch-Clamp

The F508del-CFTR chloride conductance was investigated in CFBE cells overexpressing F508del-CFTR and transfected with the following Ambion Silencer Select siRNAs against selected hit genes: siDGKG (#s3918), siGRK5 (#s6087), siLRRK1 (#s36138), STYK1 (#s30824) and siTPK1 (#s25692). siRNA selection was based on WB experiments: each gene was knocked-down with two distinct siRNAs and the one producing the largest F508del-CFTR rescue was selected. The negative control siRNA was Negl.

Cells grown on cover slips were mounted in a perfused bath on the stage of an inverted microscope (IM35, Zeiss) and kept at 37° C. The bath was perfused continuously with Ringer solution (mM: NaCl 145, KH₂PO₄ 0.4, K₂HPO₄ 1.6, D-glucose 5, MgCl₂ 1, Ca-gluconate 1.3, pH 7.4) containing 50 nM TRAM-34 at about 8 ml/min. Patch-clamp experiments were performed in the fast whole-cell configuration. Patch pipettes had an input resistance of 2-4 MΩ, and whole cell currents were corrected for serial resistance. Patch pipettes were filled with an intracellular like solution containing (mM) KCl 30, K-gluconate 95, NaH₂PO₄ 1.2, Na₂HPO₄ 4.8, EGTA 1, Ca-gluconate 0.758, MgCl₂ 1.034, D-glucose 5, ATP 3. pH was 7.2, the Ca²⁺ activity was 0.1 μM. Currents were recorded using a patch clamp amplifier (EPC 7, List Medical Electronics, Darmstadt, Germany), the LIH1600 interface and PULSE software (HEKA, Lambrecht, Germany) as well as Chart software (AD Instruments, Spechbach, Germany). In regular intervals, membrane voltages (Vc) were clamped in steps of 20 mV from −100 to +100 mV from holding potential of −100 mV. CFTR was stimulated with 25 μM Genistein and 2 μM Forskolin (Gen/Fsk). Current density was calculated by dividing whole-cell currents by cell capacitance.

### Quantitative RT-PCR

For the analysis of the 35+18 traffic regulator genes the source material was lung tissue or CFBE cells expressing wt-CFTR. The quantification of the knock-down efficiency of the 5 hit kinases used CFBE cells expressing F508del and grown as established for WB, but without CFTR corrector incubation. RNA was extracted (Trizol method—Invitrogen—or Macherey Nagel Nucleospin columns), digested with DNase I and quantified by Nanodrop spectrophotometry. Approximately 1 μg of total RNA was subjected to reverse transcription (RT) with random primers to produce cDNA (NZYTech, Lisboa, Portugal). cDNA was diluted to give a final reaction mixture concentration between 0.5 and 1.5 ng/μL. Each gene transcript was amplified in separate reactions by qRT-PCR using the SsoFast EvaGreen system (Bio-Rad, Hercules, CA), with primer sequences obtained from the Harvard primerbank or custom designed. Melt curves were checked to confirm amplification of single products, and negative controls were confirmed to be free of nonspecific amplification at 40 cycles. Products were quantified using the ΔΔCT method, normalized using as a reference gene either CAP-1 (adenylate cyclase associated protein 1) or ACTB (B actin). The correct size of amplification products was verified by agarose gel electrophoresis.

### Forskolin-Induced Swelling

Forskolin-induced swelling (FIS) assay was adapted from (Dekkers et al., 2013). Briefly, rectal organoids were seeded in a flat-bottom 96-well culture plate in 4 μL of matrigel and immersed in 50 μL of culture medium, with or without shRNA (see below) or 3 μM VX-809 (lumacaftor). After 24 h, the organoids were incubated with 3 μM calcein green (Invitrogen, USA) for 20 min and before the imaging stimulated with different concentrations of forskolin (0.02, 0.128, 0.8 and 5 μM) (Sigma-Aldrich, USA) alone or in combination with 3 μM VX-770 (ivacaftor, Selleckchem, USA). The organoids were directly analysed by confocal live cell microscopy (Leica DMI 6000B, Leica Microsystems, Germany) at 37° C. with 5% CO₂ for 60 min and images were acquired every 10 min. Organoid FIS was quantified using Cell Profiler software (Broad Institute's Imaging Platform, USA) and using image processing and in-house designed scripts (Hagemeijer et al., 2020). GraphPad Prism was used to obtain and plot values for organoid surface area. Organoid FIS is expressed as the absolute area under the curve (AUC) calculated from the normalized surface are increase (baseline=100%, t=60 min). Quantification of CFTR response to shRNAs in organoids was calculated by the difference between non-treated (Fsk) and shRNA-treated. The responses were considered as positive when the AUC was above 1000 after shRNA incubation, as described in (Dekkers et al., 2013). Each experiment was performed at least three times and each condition had technical duplicates within the same experiment.

For lentiviral transduction of human rectal organoids, lentiviral particles containing shRNAs (MISSION® shRNAs from Sigma-Aldrich) targeting the genes of interest were produced in HEK 293T. Briefly, HEK cells were transfected with 5 μg of DNA per well of a 6-well plate—2.38 μg of packaging plasmid pCMV-dR8.74psPAX2, 0.24 μg of envelop plasmid VSV-G/pMD2.G, and 2.38 μg of the vector containing the shRNA—and incubated for 18 h at 37° C., 5% CO2. The medium was replaced, and cells were incubated for 30 h at 37° C., 5% CO₂. Lentiviral particles were concentrated immediately after harvesting using the PEG-it virus precipitation solution (System Biosciences, LV810A-1). PEG-it was added to the collected medium, incubated overnight at 4° C., and afterwards centrifuged at 1500 g for 30 min. Concentrated lentiviral particles were then added to organoids split into single cells, and plates were centrifuged at 200 g for 1 h at 25° C. (spinoculation). Cells were then incubated for 6 h at 37° C., 5% CO₂. Finally, stably transduced organoids were pelleted and grown in Matrigel for expansion.

### Bioinformatic Analyses

The scores from different CFTR variants obtained at the ERQC classification screen were compared with a heatmap coupled to hierarchical clustering, using a custom R script relying on the d3heatmap package. Data was kept in the Z-score scale and was not further normalized. When building the heatmap an outlier data point (Z-score˜36) was disregarded as it biased the clustering and data visualization.

Gene ontology annotation was performed using the statistical overrepresentation test, with Bonferroni correction, available on Panther (Thomas et al., 2003). In all cases the complete term database was used. Alternatively, gene functional annotations (Gene Ontology and Reactome) were performed through the David bioinformatics resource version 6.8 (Huang da et al., 2009), also using the Bonferroni p-value correction.

Intersections of Current Data with Other CFTR-Related Datasets

The interactome processing is available online at https://github.com/hmbotelho/CFTR_interactomes

The following datasets were considered: the primary and confirmed hit genes enhancing F508del-CFTR traffic (this study), F508del-CFTR interactome in HBE41o- cells (Pankow et al., 2015), F508del-CFTR interactome in CFBE 41o- cells (Canato et al., 2018; Reilly et al., 2017), genes whose silencing significantly rescued F508del-CFTR activity (Tomati et al., 2018), candidate modifier genes of CF lung disease (Dang et al., 2020), ENaC activating genes in A549 cells (Almaça et al., 2013) and protein secretion machinery in HeLa cells (Simpson et al., 2012).

### 2. Results

### Identification of Global F508del-CFTR Traffic Regulator Genes Through High-Content Screening

In order to identify genes which increase F508del-CFTR PM levels when knocked-down an established HT fluorescence microscopy pipeline was deployed based on a Tet-inducible F508del-CFTR traffic reporter (mCherry-Flag-F508del-CFTR) stably expressed in human airway cells (Botelho et al., 2015). An N-terminus mCherry fusion provides a means of quantifying CFTR protein expression in individual cells after reverse siRNA transfection and the extracellular Flag-tag enables the detection of CFTR molecules at the PM by an anti-Flag antibody (Ab) without cell permeabilization (Botelho et al., 2015). CFTR traffic efficiency can also be quantified through the Flag/mCherry fluorescence ratio. This pipeline was used to screen the Ambion Extended Druggable Genome siRNA library (27,312 siRNAs targeting 9,128 human genes, FIG. 1A). After filtering out siRNAs with multiple or non-protein coding targets, plates with unsuccessful siRNA coating and non-representative cells (see Methods) were able to score 21,846 siRNAs, targeting 8,592 genes, regarding the F508del-CFTR traffic phenotype (FIG. 1A). In the primary screen a significant enhancement of F508del-CFTR PM levels was detected for 228 siRNAs, targeting 227 genes (Z-score5×5≥+2) (FIG. 1B,C). Only 1 gene had 2 distinct siRNAs leading to F508del-CFTR PM rescue, which was UQCRFS1 (ubiquinol-cytochrome c reductase, Rieske iron-sulfur polypeptide 1). Conversely, the primary screen also revealed that 92 siRNAs (targeting 91 genes) leading to even significantly lower levels of PM F508del-CFTR (modified Z-score≤−2). Although the HT screening pipeline (Botelho et al., 2015) also scores CFTR traffic efficiency (the anti-Flag/mCherry ratio) in each siRNA treatment, here CFTR PM levels were taken as the primary criteria for hit selection, to maximize the probability of identifying genes also leading to increased overall CFTR activity. Anyway, in the primary screen, PM levels and traffic efficiency ratios were highly correlated (R²=0.87, p<2.2×10⁻¹⁶).

To gain insight on the nature of primary screen hits, overrepresentation tests were performed on the Gene Ontology (GO) terms associated with the positive hit genes, i.e., siRNAs enhancing F508del-CFTR traffic the same analysis was also performed with the negative hit genes. Considering the genes targeted by enhancer siRNAs, an overrepresentation of genes associated with the PM (cell component), kinase activity, ionic binding and transport and adenylyl nucleotide binding (molecular function) and phosphorylation (biological process) among the positive hit genes was observed. These terms suggest that several of the positive hit genes from the primary screen are other PM proteins, possibly competing with CFTR for the secretory pathway machinery. Regarding genes targeted by siRNAs resulting in lower F508del-CFTR PM levels, GO revealed an enrichment in PM proteins (cellular component), enzymes and metal binding, pointing to biological pathways with possible favourable effects on F508del-CFTR traffic.

Identification of Top Hits by siRNA Secondary Screening

We sought to validate the primary screen hits by rescreening the positive hit genes using distinct siRNA molecules. This secondary screen consisted of a library of 450 siRNAs targeting the 225 hit genes of the primary screen, whose impact on F508del-CFTR PM levels was scored by the same assay (FIG. 2A). Considering both the primary and secondary screens, 35 genes targeted by 2 distinct siRNAs were identified with robust increases in F508del-CFTR PM levels, i.e., above the hit threshold, in both screening rounds. This stringent criterion equates to a 15% hit validation rate. These genes, which were termed ‘confirmed hits’, when inhibited significantly increase the availability of F508del-CFTR at the PM (FIG. 2B, C) being thus potential F508del-CFTR retention factors. Notably, in the screening assay these confirmed hits rescue F508del-CFTR to a larger extent than the clinical drug VX-809 (lumacaftor, 3 μM). Enrichment analysis of these 35 hit genes did not reveal any overrepresentation of GO terms, likely due to the small sample size.

To determine the physiological relevance of these 35 confirmed hits their expression in human native lung tissue and CFBE expressing wt-CFTR was measured by RT-PCR. With the exception of 4 genes (CITED2, KIF17, ACSBG2, GUCY2D), all were found to be expressed in human native lung tissue, thus showing that ˜89% of the top hits were physiologically relevant. Expression of 3 genes (APOB, ACSBG2, GUCY2D) could not be detected in CFBE cells.

### Validation of Top F508del-CFTR Traffic Regulators by Processing and Functional Assessment

Since the 35 confirmed hits were defined solely by microscopy-based experiments, complementary assays were performed to validate them as F508del-CFTR regulators. Initially, Western blotting (WB) was used as a readout for CFTR processing and ER exit to confirm the effect of gene KDs on CFTR PM levels. CFBE cells expressing untagged F508del-CFTR were transfected with siRNAs targeting each of the 35 confirmed microscopy hits and assessed F508del-CFTR processing (FIG. 3). These experiments validated 32 of the 35 genes (91%) as putative regulators of F508del-CFTR targeting to the PM (statistically significant band C increases over control with at least 1 siRNA). The remarkable validation rate prompted us to examine whether the genes targeted by sRNAs which scored just below the hit threshold in the secondary microscopy screen could also rescue F508del-CFTR processing in a meaningful manner. Therefore, identical WB assays were performed targeting 18 additional genes which were not confirmed in the secondary screen. Knocking down all 18 genes significantly enhanced band C versus the control, indicating a greater sensitivity of WB analyses, albeit at much lower throughput. Interestingly, among the 50 genes validated in the WB assay, 5 are kinases: LRRK1, TPK1, STYK1, GRK5 and DGKG, of which the LRRK1, TPK1 and STYK1 KDs produced F508del-CFTR rescue in the HT microscopy and WB assays.

To determine whether the 35 confirmed hit genes also rescue F508del-CFTR chloride conductance, the halide-sensitive YFP (HS-YFP) fluorescence quenching assay was performed in combination with the same siRNA KDs as in the secondary microscopy screen. Cells expressing wt-CFTR were also tested. The assay validated 20 genes targeted by 26 siRNAs that significantly enhance F508del-CFTR function above control (Z-score>+1), including LRRK1 and STYK1, but not TPK1 (FIG. 4). KD of 10 of the 20 genes enhanced F508del- but not wt-CFTR function: CITED2, CLDN4, COL5A1, CREBBP, DCSTAMP, FOLR2, LDLRAD3, PCDHB2, RECQL5 and ZNF384. Conversely, only 2 gene KDs enhance wt- but not F508del-CFTR function: QRSL1 and TPK1.

Top Traffic Regulators have Mixed Effects at ERQC Folding Checkpoints

As our approach aimed at identifying proteins which retain F508del-CFTR intracellularly, some of the primary hits may be constituents of the ERQC checkpoints. To test this, the effects of their KD on the traffic of: i) wt-CFTR; ii), F508del-CFTR revertant mutations (F508del-G550E-CFTR, F508del-R1070W-CFTR, F508del-4RK-CFTR); or iii) a traffic-inhibiting CFTR mutant (DD/AA-CFTR) was assessed, all stably expressed as Tet-inducible traffic reporters in CFBE cell lines. This “classification screen” aimed at assigning each hit gene to one or more ERQC checkpoints (FIG. 5A-C). The 5 cell lines were reverse transfected with 450 siRNAs targeting the 225 primary screen hit genes and a positive effect was considered when at least 1 siRNA significantly increased CFTR PM levels (Z-score>+1. Treatment with VX-809 was used in parallel as a positive control. When excluding control siRNAs a positive effect for 22 genes for wt-CFTR, 52 genes for F508del-G550E-CFTR, 20 genes for F508del-R1070W-CFTR, 25 genes for F508del-4RK-CFTR and 38 genes for DD/AA-CFTR (FIG. 5A) was obtained. It was not clear how to interpret the MoA of the genes which were not hits for F508del-CFTR because even revertant mutants which target the same ERQC checkpoint (F508del-G550E and F508del-R1070Q) diverged significantly in the number of hit genes.

We therefore focused on the 35 confirmed hits enhancing F508del-CFTR PM and performed hierarchical clustering of the classification screen data (FIG. 5C). 7 clusters were identified, grouping siRNAs which produced the following phenotypes: Cluster 1) very strong traffic inhibition of wt-CFTR (only contains the NTNG2 gene); Cluster 2) strongly inhibit wt- and F508del-R1070W-CFTR traffic, and have mixed effects on the other CFTR variants; Cluster 3) inhibit wt- (not as strongly as Cluster 1), F508del-R1070W-, F508del-4RK- and F508del-G550E-CFTR traffic, with mixed effects on DD/AA-CFTR; Cluster 4) strongly enhance F508del-G550E, and DD/AA-CFTR traffic, strongly inhibits wt-CFTR traffic and has negligible effects on the two remaining variants (only contains the LIN9 gene); Cluster 5) mildly decrease wt- and F508del-R1070W-CFTR traffic and have mixed effects on other variants; Cluster 6) enhance traffic of all variants; Cluster 7) strongly enhance wt- and F508del-R1070W traffic.

This classification suggests that genes in Clusters 1 and 2 are highly specific F508del-CFTR traffic regulators, while those in Cluster 3 genes are less specific, given their milder phenotypes on the F508del-CFTR variants and reduced potency when combined with VX-809. The rescue of F508del-CFTR PM localization by siRNAs targeting Cluster 5 genes seem to be additive with NBD1 folding, given their enhanced rescue when combined with either VX-809 (siSTYK1, siISL2, siLRRK1) or G550E (siLDLRAD3, siCYSLTR2, siOXSR1, siNOVA1, siZNF384), a revertant proposed to stabilize NBD1 and its dimerization. Cluster 6 contains genes whose KD leads to overall increased CFTR traffic. Overall, inter-cluster differences are generally small, which was interpreted as the gene KDs targeting non-ERQC pathways or having mixed effects, targeting more than one checkpoint. Nevertheless, the kinases in this gene set (LRRK1, TPK1 and STYK1) seem to be specific F508del-CFTR regulators, given that they only achieve hit status in this mutant (either alone or in combination with VX-809), with the notable exception of LRRK1 in F508del-G550E-CFTR (FIG. 5B).

### F508del-CFTR Traffic Screen Hits are a Novel Category of CFTR Regulators

To ascertain the overall pathways targeted by F508del-CFTR traffic regulators identified here, the primary and secondary screen hits from this study were compared with published datasets of CFTR interactors and regulators or CF disease modulators (FIG. 5D): F508del-CFTR interactome in HBE41o- cells (Pankow et al., 2015), F508del-CFTR interactome in CFBE 41o- cells (Canato et al., 2018; Reilly et al., 2017), genes whose silencing significantly rescued F508del-CFTR activity (Tomati et al., 2018), candidate modifier genes of CF lung disease (Dang et al., 2020), ENaC activating genes in A549 cells (Almaça et al., 2013) and protein secretion machinery in HeLa cells (Simpson et al., 2012). 221 instances were found where the same gene was mentioned in one pair of the published datasets (gray lines). When inspecting our primary (outer track) and secondary screen hits (inner track), there were 32 instances where some of the genes were mentioned in other studies (lines in shades of red). Amongst the confirmed hits there are only 3 which appear in any of the other datasets: ARVF (Dang), COL5A1 (Reilly) and OXSR1 (Simpson). When considering the 5 kinases within the primary and secondary hits, only GRK5 can be found in one of the other datasets (Almaça), indicating that knocking down GRK5 may not only recue F508del-CFTR traffic but also inhibit ENaC activity, both of which are desirable for reversing CF symptoms. The low amount of genes which are common to this study and this dataset selection suggests that our HT screening platform is sensitive to identify CFTR regulators which may interact with CFTR indirectly or not be part of canonical CFTR regulatory networks.

Selection of kinases among top hits as potential drug targets Since the aim was to identify druggable targets enhancing F508del-CFTR traffic, 5 kinases were selected among the top hits for further studies: LRRK1, TPK1, STYK1, GRK5 and DGKG (FIG. 1A). LRRK1, TPK1 and STYK1 were hits in the primary and secondary traffic screen, as well on the WB validation. GRK5 and DGKG were hits only on the primary traffic screen but a robust F508del-CFTR rescue on WB was observed. Their KD produced high average PM scores in the primary screen and in processing assessed by WB (18- and 12.3-fold change, respectively. The motivation for this selection was the amenability of kinases to small-molecule inhibition, which makes them good drug targets. Reverse transfection of siRNAs targeting these genes in CFBE cells expressing non-tagged F508del-CFTR with and without combined administration of VX-809 (3 μM), VX-661 (5 μM) and VX-661 plus VX-445 (3 μM) (FIG. 6) was performed. Knock-down efficiency of each kinase mRNA was found to be between 22 and 60% (FIG. 7). Band C rescue was additive with VX-809 and VX-661 for GRK5 and LRRK1, and unaffected by DGKG KD. TPK1 KD reduced band C levels in the presence of correctors (FIG. 6A, B). Most interestingly, the effects of all siRNAs were potentiated when cells were treated with the clinical corrector VX-445 in addition to VX-661, except GRK5, which did not affect band C, even though band B levels were reduced when comparing VX-661 plus VX-445 versus the DMSO control (FIG. 6E). These data are suggestive of some overlap in the pathways targeted by the kinases other than LRRK1 (which is always additive) and the corrector molecules, suggesting possibly a common MoA.

To increase the pharmacological interest of the 5 selected kinases, the effect of their inhibition on the restoration of F508del-CFTR function was tested by patch-clamp. The KD of 4 out of the 5 kinases (STYK1, TPK1, GRK5 and LRRK1) rescued F508del-CFTR function, being GRK5 and LRRK1 the most effective and DGKG had no effect. To complement these observations, the functional rescue of F508del-CFTR on the forskolin-induced swelling (FIS) assay performed with intestinal organoids was also tested while knocking down the 5 hit kinases. Using a lentiviral vector encoding appropriate shRNAs it was only possible to successfully KD LRRK1, STYK1 and GRK5. Only the LRRK1 KD was effective in rescuing the swelling phenotype.

To determine whether these kinases act on the same or distinct pathways/ERQC checkpoints, the combined effects of their siRNAs on rescuing F508del-CFTR revertants (F508del-G550E, F508del-R1070W, F508del-4RK) or the DD/AA-CFTR traffic mutant were analysed.

Kinase KDs produced a complex response in the F508del-G550E and F508del-R1070W revertants, which target the first ERQC checkpoint. Knocking down LRRK1 or STYK1 does not significantly affect band C levels in most situations, either alone or in the presence of correctors, indicating that these KDs may act at the level of this checkpoint. The DGKG and GRK5 KDs are usually additive, but VX-445 has a deleterious effect on band C rescue when knocking down TPK1, DGKG or GRK5 on F508del-G550E-CFTR, suggesting a common MoA. The F508del-4RK-CFTR revertant was additive to all kinase KDs, alone or in the presence of correctors, except for TPK1 and GRK5, which produced unchanged (TPK1) or lower (GRK) band C levels. The DD/AA traffic mutant was not affected.

Finally, the specificity of CFTR traffic rescue through hit kinases KD was determined by performing kinase KDs in non-F508del-CFTR class II mutants: R560S, G85E and N1303K. No traffic rescue was observed, even in combination with CFTR folding correctors, indicating that the 5 kinases specifically regulate F508del-CFTR traffic.

### GRK5 Activity Regulates F508del-CFTR PM Stability

We selected GRK5 (G Protein-Coupled Receptor Kinase 5) for additional mechanistic studies because it was the hit kinase with best performance in rescuing F508del-CFTR in most assays, including patch-clamp and also because of its possible overlap with the pathway targeted by the VX-445 drug. Moreover, it has been proposed as a therapeutic target for cardiovascular disorders (Lieu and Koch, 2019), making it an attractive target for drug repurposing. GRK5 is involved in the transduction of ligand binding and desensitization of beta2-adrenergic receptor (β2AR). Upon binding of an agonist, GRK5 is recruited to bind and phosphorylate β2AR, thereby contributing to receptor internalization and signal termination (Pfleger et al., 2019). Importantly, β2AR is involved in endogenous CFTR activation in the epithelium (Naren et al., 2003) as its activation increases cellular cAMP levels. This provides a possible mechanistic connection between GRK5 and CFTR, which needs to be clarified in order to validate this gene as a new druggable target. Recently, the first GRK5-specific inhibitor was described: (R,Z)-3-((4-(2-Bromoacetamido)-3,5-dimethyl-1H-pyrrol-2-yl)-methylene)-2-oxo-N-(1-phenylethyl)indoline-5-carboxamide (FIG. 8), a selective covalent GRK5 Inhibitor (IC50=8.6 nM) with 1400-fold selectivity against GRK2 (Rowlands et al., 2021).

To complement our siRNA-based data and to determine whether F508del-CFTR rescue could be obtained by inhibiting GRK5 with (R,Z)-3-((4-(2-Bromoacetamido)-3,5-dimethyl-1H-pyrrol-2-yl)-methylene)-2-oxo-N-(1-phenylethyl)indoline-5-carboxamide, a dose-response WB assay was performed. CFBE cells expressing F508del-CFTR were incubated with (R,Z)-3-((4-(2-Bromoacetamido)-3,5-dimethyl-1H-pyrrol-2-yl)-methylene)-2-oxo-N-(1-phenylethyl)indoline-5-carboxamide [0-1 μM] and band C levels were quantified and compared with treatment with VX-661 (FIG. 9A). A statistically significant band C rescue was observed at 0.3 and 1.0 μM (50% of VX-661, FIG. 9B), albeit without improved CFTR processing, due to a proportional increase in band B (FIG. 9C). Higher (R,Z)-3-((4-(2-Bromoacetamido)-3,5-dimethyl-1H-pyrrol-2-yl)-methylene)-2-oxo-N-(1-phenylethyl)indoline-5-carboxamide concentrations could not be tested due to cellular toxicity (not shown).

To ascertain whether (R,Z)-3-((4-(2-Bromoacetamido)-3,5-dimethyl-1H-pyrrol-2-yl)-methylene)-2-oxo-N-(1-phenylethyl)indoline-5-carboxamide is additive to CFTR correctors, CFBE cells expressing F508del-CFTR were incubated with 1 μM (R,Z)-3-((4-(2-Bromoacetamido)-3,5-dimethyl-1H-pyrrol-2-yl)-methylene)-2-oxo-N-(1-phenylethyl)indoline-5-carboxamide in the presence and absence of VX-661, VX-445 or VX-661 plus VX-445. When performing a WB analysis (FIG. 9D-F) additivity of (R,Z)-3-((4-(2-Bromoacetamido)-3,5-dimethyl-1H-pyrrol-2-yl)-methylene)-2-oxo-N-(1-phenylethyl)indoline-5-carboxamide to all corrector treatments was observed, indicating a distinct MoA. Similarly, HS-YFP quenching studies were performed which also showed additivity in all conditions (FIG. 9G, H).

These results suggest that not only can F508del-CFTR traffic be rescued by inhibiting GRK5 with (R,Z)-3-((4-(2-Bromoacetamido)-3,5-dimethyl-1H-pyrrol-2-yl)-methylene)-2-oxo-N-(1-phenylethyl) indoline-5-carboxamide, but also that the rescue obtained with clinical correctors can be maximized by co-administrating (R,Z)-3-((4-(2-Bromoacetamido)-3,5-dimethyl-1H-pyrrol-2-yl)-methylene)-2-oxo-N-(1-phenylethyl)indoline-5-carboxamide.

This description is of course not in any way restricted to the forms of implementation presented herein and any person with an average knowledge of the area can provide many possibilities for modification thereof without departing from the general idea as defined by the claims. The preferred forms of implementation described above can obviously be combined with each other. The following claims further define the preferred forms of implementation.

### REFERENCES

(2021). Cystic Fibrosis Mutation Database.
Almaca, J., Dahimene, S., Appel, N., Conrad, C., Kunzelmann, K., Pepperkok, R., and Amaral, M. D. (2011). Functional genomics assays to study CFTR traffic and ENaC function. Methods Mol Biol 742, 249-264. 10.1007/978-1-61779-120-8_15.
Almaça, J., Faria, D., Sousa, M., Uliyakina, I., Conrad, C., Sirianant, L., Clarke, L. A., Martins, J. P., Santos, M., Hériché, J.-K., et al. (2013). High-content siRNA screen reveals global ENaC regulators and potential cystic fibrosis therapy targets. Cell 154, 1390-1400. 10.1016/j.cell.2013.08.045.
Amaral, M. D. (2004). CFTR and chaperones: processing and degradation. J Mol Neurosci 23, 41-48. 10.1385/JMN:23:1-2:041.
Amaral, M. D. (2021). How to determine the mechanism of action of CFTR modulator compounds: A gateway to theranostics. Eur J Med Chem 210, 112989. 10.1016/j.ejmech.2020.112989.
Amaral, M. D., and Farinha, C. M. (2013). Rescuing mutant CFTR: a multi-task approach to a better outcome in treating cystic fibrosis. Curr Pharm Des 19, 3497-3508.
Amaral, M. D., Farinha, C. M., Matos, P., and Botelho, H. M. (2016). Investigating Alternative Transport of Integral Plasma Membrane Proteins from the ER to the Golgi: Lessons from the Cystic Fibrosis Transmembrane Conductance Regulator (CFTR). Methods Mol Biol 1459, 105-126. 10.1007/978-1-4939-3804-9_7.
Boccella, N., Paolillo, R., and Perrino, C. (2019). Epac1 inhibition as a novel cardioprotective strategy: lights and shadows on GRK5 canonical and non-canonical functions. Cardiovasc Res 115, 1684-1686. 10.1093/cvr/cvz188.
Botelho, H. M., Uliyakina, I., Awatade, N. T., Proenca, M. C., Tischer, C., Sirianant, L., Kunzelmann, K., Pepperkok, R., and Amaral, M. D. (2015). Protein traffic disorders: an effective high-throughput fluorescence microscopy pipeline for drug discovery. Sci Rep 5, 9038. 10.1038/srep09038.
Canato, S., Santos, J. D., Carvalho, A. S., Aloria, K., Amaral, M. D., Matthiesen, R., Falcao, A. O., and Farinha, C. M. (2018). Proteomic interaction profiling reveals KIFC1 as a factor involved in early targeting of F508del-CFTR to degradation. Cell Mol Life Sci 75, 4495-4509. 10.1007/s00018-018-2896-7.
Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). CellProfiler: image analysis software for identifying and quantifying cell phenotypes. Genome Biol 7, R100. 10.1186/gb-2006-7-10-r100.
Dalemans, W., Barbry, P., Champigny, G., Jallat, S., Dott, K., Dreyer, D., Crystal, R. G., Pavirani, A., Lecocq, J. P., and Lazdunski, M. (1991). Altered chloride ion channel kinetics associated with the delta F508 cystic fibrosis mutation. Nature 354, 526-528. 10.1038/354526a0.
Dang, H., Polineni, D., Pace, R. G., Stonebraker, J. R., Corvol, H., Cutting, G. R., Drumm, M. L., Strug, L. J., O'Neal, W. K., and Knowles, M. R. (2020). Mining GWAS and eQTL data for CF lung disease modifiers by gene expression imputation. PLoS One 15, e0239189. 10.1371/journal.pone.0239189.
Dekkers, J. F., Wiegerinck, C. L., de Jonge, H. R., Bronsveld, I., Janssens, H. M., de Winter-de Groot, K. M., Brandsma, A. M., de Jong, N. W. M., Bijvelds, M. J. C., Scholte, B. J., et al. (2013). A functional CFTR assay using primary cystic fibrosis intestinal organoids. Nature Medicine 19, 939-945. 10.1038/nm.3201.
Denning, G. M., Anderson, M. P., Amara, J. F., Marshall, J., Smith, A. E., and Welsh, M. J. (1992). Processing of mutant cystic fibrosis transmembrane conductance regulator is temperature-sensitive. Nature 358, 761-764. 10.1038/358761a0.
Erfle, H., Neumann, B., Liebel, U., Rogers, P., Held, M., Walter, T., Ellenberg, J., and Pepperkok, R. (2007). Reverse transfection on cell arrays for high content screening microscopy. Nat Protoc 2, 392-399. 10.1038/nprot.2006.483.
Farinha, C. M., and Amaral, M. D. (2005). Most F508del-CFTR is targeted to degradation at an early folding checkpoint and independently of calnexin. Mol Cell Biol 25, 5242-5252. 10.1128/MCB.25.12.5242-5252.2005.
Farinha, C. M., and Canato, S. (2017). From the endoplasmic reticulum to the plasma membrane: mechanisms of CFTR folding and trafficking. Cell Mol Life Sci 74, 39-55. 10.1007/s00018-016-2387-7.
Farinha, C. M., King-Underwood, J., Sousa, M., Correia, A. R., Henriques, B. J., Roxo-Rosa, M., Da Paula, A. C., Williams, J., Hirst, S., Gomes, C. M., et al. (2013a). Revertants, Low Temperature, and Correctors Reveal the Mechanism of F508del-CFTR Rescue by VX-809 and Suggest Multiple Agents for Full Correction. Chem Biol 20, 943-955. 10.1016/j.chembiol.2013.06.004.
Farinha, C. M., Matos, P., and Amaral, M. D. (2013b). Control of cystic fibrosis transmembrane conductance regulator membrane trafficking: not just from the endoplasmic reticulum to the Golgi. FEBS J 280, 4396-4406. 10.1111/febs.12392.
Fiedorczuk, K., Chen, J. (2022). Mechanism of CFTR correction by type I folding correctors. Cell 185, 158-168. 10.1016/j.cell.2021.12.009.
Hagemeijer, M. C., Vonk, A. M., Awatade, N. T., Silva, I. A. L., Tischer, C., Hilsenstein, V., Beekman, J. M., Amaral, M. D., and Botelho, H. M. (2020). An open-source high-content analysis workflow for CFTR function measurements using the forskolin-induced swelling assay. Bioinformatics. 10.1093/bioinformatics/btaa1073.
He, L., Kota, P., Aleksandrov, A. A., Cui, L., Jensen, T., Dokholyan, N. V., and Riordan, J. R. (2013). Correctors of DeltaF508 CFTR restore global conformational maturation without thermally stabilizing the mutant protein. FASEB J 27, 536-545. 10.1096/fj.12-216119.
Homan, K. T., Waldschmidt, H. V., Glukhova, A., Cannavo, A., Song, J., Cheung, J. Y., Koch, W. J., Larsen, S. D., and Tesmer, J. J. G. (2015). Crystal Structure of G Protein-coupled Receptor Kinase 5 in Complex with a Rationally Designed Inhibitor. J Biol Chem 290, 20649-20659. 10.1074/jbc.M115.647370.
Huang da, W., Sherman, B. T., and Lempicki, R. A. (2009). Systematic and integrative analysis of large gene lists using DAVID bioinformatics resources. Nat Protoc 4, 44-57. 10.1038/nprot.2008.211.
Lieu, M., and Koch, W. J. (2019). GRK2 and GRK5 as therapeutic targets and their role in maladaptive and pathological cardiac hypertrophy. Expert Opin Ther Targets 23, 201-214. 10.1080/14728222.2019.1575363.
Lopes-Pacheco, M., Silva, I. A. L., Turner, M. J., Carlile, G. W., Sondo, E., Thomas, D. Y., Pedemonte, N., Hanrahan, J. W., and Amaral, M. D. (2020). Characterization of the mechanism of action of RDR01752, a novel corrector of F508del-CFTR. Biochem Pharmacol 180, 114133. 10.1016/j.bcp.2020.114133.
MacKenzie, T., Gifford, A. H., Sabadosa, K. A., Quinton, H. B., Knapp, E. A., Goss, C. H., and Marshall, B. C. (2014). Longevity of patients with cystic fibrosis in 2000 to 2010 and beyond: survival analysis of the Cystic Fibrosis Foundation patient registry. Ann Intern Med 161, 233-241. 10.7326/M13-0636.
Middleton, P. G., Mall, M. A., Drevinek, P., Lands, L. C., McKone, E. F., Polineni, D., Ramsey, B. W., Taylor-Cousar, J. L., Tullis, E., Vermeulen, F., et al. (2019). Elexacaftor-Tezacaftor-Ivacaftor for Cystic Fibrosis with a Single Phe508del Allele. N Engl J Med 381, 1809-1819. 10.1056/NEJMoa1908639.
Moniz, S., Sousa, M., Moraes, B. J., Mendes, A. I., Palma, M., Barreto, C., Fragata, J. I., Amaral, M. D., and Matos, P. (2013). HGF stimulation of Rac1 signaling enhances pharmacological correction of the most prevalent cystic fibrosis mutant F508del-CFTR. ACS Chem Biol 8, 432-442. 10.1021/cb300484r.
Naren, A. P., Cobb, B., Li, C., Roy, K., Nelson, D., Heda, G. D., Liao, J., Kirk, K. L., Sorscher, E. J., Hanrahan, J., et al. (2003). A macromolecular complex of beta 2 adrenergic receptor, CFTR, and ezrin/radixin/moesin-binding phosphoprotein 50 is regulated by PKA. Proc Natl Acad Sci USA 100, 342-346. 10.1073/pnas.0135434100.
Neumann, B., Walter, T., Hériché, J.-K., Bulkescher, J., Erfle, H., Conrad, C., Rogers, P., Poser, I., Held, M., Liebel, U., et al. (2010). Phenotypic profiling of the human genome by time-lapse microscopy reveals cell division genes. Nature 464, 721-727. 10.1038/nature08869.
Nishimura, N., and Balch, W. E. (1997). A di-acidic signal required for selective export from the endoplasmic reticulum. Science 277, 556-558.
Okiyoneda, T., Veit, G., Dekkers, J. F., Bagdany, M., Soya, N., Xu, H., Roldan, A., Verkman, A. S., Kurth, M., Simon, A., et al. (2013). Mechanism-based corrector combination restores DeltaF508-CFTR folding and function. Nat Chem Biol 9, 444-454. 10.1038/nchembio.1253.
Pankow, S., Bamberger, C., Calzolari, D., Martínez-Bartolomé, S., Lavallee-Adam, M., Balch, W. E., and Yates, J. R., 3rd (2015). AF508 CFTR interactome remodelling promotes rescue of cystic fibrosis. Nature 528, 510-516. 10.1038/nature15729.
Pedemonte, N., Zegarra-Moran, O., and Galietta, L. J. (2011). High-throughput screening of libraries of compounds to identify CFTR modulators. Methods Mol Biol 741, 13-21. 10.1007/978-1-61779-117-8_2.
Perkins, L. A., Fisher, G. W., Naganbabu, M., Schmidt, B. F., Mun, F., and Bruchez, M. P. (2018). High-Content Surface and Total Expression siRNA Kinase Library Screen with VX-809 Treatment Reveals Kinase Targets that Enhance F508del-CFTR Rescue. Mol Pharm 15, 759-767. 10.1021/acs.molpharmaceut.7b00928.
Pfleger, J., Gresham, K., and Koch, W. J. (2019). G protein-coupled receptor kinases as therapeutic targets in the heart. Nat Rev Cardiol 16, 612-622. 10.1038/s41569-019-0220-3.
Reilly, R., Mroz, M. S., Dempsey, E., Wynne, K., Keely, S. J., McKone, E. F., Hiebel, C., Behl, C., and Coppinger, J. A. (2017). Targeting the PI3K/Akt/mTOR signalling pathway in Cystic Fibrosis. Sci Rep 7, 7642. 10.1038/s41598-017-06588-z.
Riordan, J. R., Rommens, J. M., Kerem, B., Alon, N., Rozmahel, R., Grzelczak, Z., Zielenski, J., Lok, S., Plavsic, N., Chou, J. L., et al. (1989). Identification of the cystic fibrosis gene: cloning and characterization of complementary DNA. Science 245, 1066-1073.
Rowlands, R. A., Chen, Q., Bouley, R. A., Avramova, L. V., Tesmer, J. J. G., and White, A. D. (2021). Generation of Highly Selective, Potent, and Covalent G Protein-Coupled Receptor Kinase 5 Inhibitors. J Med Chem 64, 566-585. 10.1021/acs.jmedchem.0c01522.
Simpson, J. C., Joggerst, B., Laketa, V., Verissimo, F., Cetin, C., Erfle, H., Bexiga, M. G., Singan, V. R., Heriche, J. K., Neumann, B., et al. (2012). Genome-wide RNAi screening identifies human proteins with a regulatory function in the early secretory pathway. Nat Cell Biol 14, 764-774. 10.1038/ncb2510.
Sondo, E., Tomati, V., Caci, E., Esposito, A. I., Pfeffer, U., Pedemonte, N., and Galietta, L. J. (2011). Rescue of the mutant CFTR chloride channel by pharmacological correctors and low temperature analyzed by gene expression profiling. Am J Physiol Cell Physiol 301, C872-885. 10.1152/ajpcell.00507.2010.
Taylor-Cousar, J. L., Munck, A., McKone, E. F., van der Ent, C. K., Moeller, A., Simard, C., Wang, L. T., Ingenito, E. P., McKee, C., Lu, Y., et al. (2017). Tezacaftor-Ivacaftor in Patients with Cystic Fibrosis Homozygous for Phe508del. N Engl J Med 377, 2013-2023. 10.1056/NEJMoa1709846.
Thomas, P. D., Campbell, M. J., Kejariwal, A., Mi, H., Karlak, B., Daverman, R., Diemer, K., Muruganujan, A., and Narechania, A. (2003). PANTHER: a library of protein families and subfamilies indexed by function. Genome Res 13, 2129-2141. 10.1101/gr.772403.
Tomati, V., Pesce, E., Caci, E., Sondo, E., Scudieri, P., Marini, M., Amato, F., Castaldo, G., Ravazzolo, R., Galietta, L. J. V., et al. (2018). High-throughput screening identifies FAU protein as a regulator of mutant cystic fibrosis transmembrane conductance regulator channel. J Biol Chem 293, 1203-1217. 10.1074/jbc.M117.816595.
Traynham, C. J., Hullmann, J., and Koch, W. J. (2016). “Canonical and non-canonical actions of GRK5 in the heart”. J Mol Cell Cardiol 92, 196-202. 10.1016/j.yjmcc.2016.01.027.
Wang, X., Matteson, J., An, Y., Moyer, B., Yoo, J. S., Bannykh, S., Wilson, I. A., Riordan, J. R., and Balch, W. E. (2004). COPII-dependent export of cystic fibrosis transmembrane conductance regulator from the ER uses a di-acidic exit code. J Cell Biol 167, 65-74. 10.1083/jcb.200401035.
Yarwood, R., Hellicar, J., Woodman, P. G., and Lowe, M. (2020). Membrane trafficking in health and disease. Dis Model Mech 13, dmm043448. 10.1242/dmm.043448.
Younger, J. M., Chen, L., Ren, H. Y., Rosser, M. F., Turnbull, E. L., Fan, C. Y., Patterson, C., and Cyr, D. M. (2006). Sequential quality-control checkpoints triage misfolded cystic fibrosis transmembrane conductance regulator. Cell 126, 571-582. 10.1016/j.cell.2006.06.041.

1. A method of identifying agents for the treatment of Cystic Fibrosis caused by the mutation F508del in the gene encoding the transmembrane conductance regulator protein (F508del-CFTR) comprising the steps of:
a) contacting a candidate agent with at least one gene sequence selected from SEQ ID Nos: 228 to 478 or with at least one of its protein products of SEQ ID Nos: 1 to 227 as therapeutic targets;
b) determining whether the candidate agent modulates the activity of the F508del-CFTR protein and its plasma membrane traffic.

2. Method according to claim 1, wherein at least one gene sequence is selected from the group consisting of SEQ ID Nos: 232, 237, 238, 250, 253, 257, 259, 263, 276, 285, 296, 297, 301, 317, 318, 319, 333, 338, 369, 378, 396, 401, 405, 409, 410, 411, 417, 420, 421, 430, 442, 450, 458, 459, 471, 472 and 478.

3. Method according to claim 1, wherein activity modulation is affected by inhibiting gene expression of at least one gene sequence selected from SEQ ID Nos: 228 to 478 or the activity of at least one of its protein products of SEQ ID Nos: 1 to 227.

4. Method according to claim 1, wherein the candidate agents identified inhibit the activity of at least one gene sequence selected from SEQ ID Nos: 228 to 478 or the activity of at least one of its protein products of SEQ ID Nos: 1 to 227 by more than 20% relative to the activity in the absence of the candidate agent.

5. Method according to claim 1, wherein inhibition is affected by reducing gene expression of at least one gene sequence selected from Seq.ID: 228 to 478.

6. Method according to claim 1, wherein the activity is inhibited on the protein level of at least one protein sequence selected from SEQ ID Nos: 1 to 227.

7. Method according to claim 1, wherein the ability of the candidate agent to bind to a protein product, or a domain thereof, from at least one gene sequence selected from SEQ ID Nos: 228 to 478 or from at least one protein sequence from SEQ ID Nos: 1 to 227 is determined.

8. Method according to claim 1, wherein the gene sequence protein products are TPK1, LRRK1, STYK1, GRK5 or DGKG.

9. Method according to claim 1, wherein (R,Z)-3-((4-(2-Bromoacetamido)-3,5-dimethyl-1H-pyrrol-2-yl)-methylene)-2-oxo-N-(1-phenylethyl)indoline-5-carboxamide is the candidate agent.

10. Method according to claim 1, wherein the method is carried out as a high-throughput assay.

11. Method according to claim 1, wherein the method is carried out as a cell-based assay.

12. An agent for use in the treatment of Cystic Fibrosis caused by the mutation F508del in the gene encoding the CFTR protein identified by the method of claim 1.

13. The agent according to claim 12, wherein the agent is (R,Z)-3-((4-(2-Bromoacetamido)-3,5-dimethyl-1H-pyrrol-2-yl)-methylene)-2-oxo-N-(1-phenylethyl)indoline-5-carboxamide.

14. A pharmaceutical composition comprising the agent of claim 12.

15. The pharmaceutical composition according to claim 14 further comprising at least one pharmaceutically acceptable diluent, carrier, adjuvant or an excipient.
